(19) United States
(12) Patent Application Publication
(10) Pub. No.: US 2022/0074929 A1
(43) Pub. Date: Mar. 10, 2022

(54) QUANTIFICATION, ISOLATION, AND CHARACTERIZATION OF EXOSOMES USING DROPLET-BASED AND WELL-BASED MICROFLUIDIC SYSTEMS

(71) Applicant: THE HONG KONG UNIVERSITY OF SCIENCE AND TECHNOLOGY, Hong Kong (CN)

(72) Inventors: Shuhuai YAO, Hong Kong (CN); Lei ZHENG, Guangzhou (CN); Xiaonan XU, Hong Kong (CN); Chunchen LIU, Hong Kong (CN); Yu HU, Hong Kong (CN)

(21) Appl. No.: 16/625,282

(22) PCT Filed: Oct. 11, 2018
(86) PCT No.: PCT/CN2018/109760

§ 371 Date: Dec. 20, 2019

Related U.S. Application Data
(60) Provisional application No. 62/606,687, filed on Oct. 5, 2017.

(51) Int. Cl. G01N 33/543; G01N 33/574

(52) U.S. Cl. CPC G01N 33/54326; G01N 33/57488

(57) ABSTRACT

Methods of quantification, isolation, and characterization of exosomes are provided. Exosomes can be quantified by contacting a sample with a capture bead comprising a bead and a first binding agent, and a second binding agent. The first binding agent binds to a first biomolecule in the exosomes to produce a first complex and the second binding agent binds to a second biomolecule in the exosomes of the first complex to produce a second complex. The first complexes and the second complexes are quantified based on a detectable signal conjugated to the second binding agent. A microwell or a droplet generation is utilized to quantify the first complexes and the second complexes. Quantifying the exosomes is used to diagnose a cancer in a subject. In such methods, the first and the second binding agents bind to cancer biomarkers present in the exosomes.

### CROSS-REFERENCE TO RELATED APPLICATION

This application is the U.S. national stage application of International Patent Application No. PCT/CN2018/109760, filed Oct. 11, 2018; which claims the benefit of U.S. Provisional Patent Application Ser. No. 62/606,687, filed Oct. 5, 2017, the disclosure of each of which is hereby incorporated by reference in its entirety, including any figures, tables, or drawings.

### BACKGROUND OF THE INVENTION

Exosomes are proposed as potent biomarkers for cancer diagnostics. Exosomes are non-uniform membranous particles with a diameter of 30-150 nm secreted from cells through plasma membrane fusion of multivesicular bodies (MVBs). Exosomes shed from tumor tissues carry numerous biomarkers such as transmembrane and cytosolic proteins (CD9, CD63, CD81, etc.), lipids, DNA and microRNA. Specific proteins, such as Glypican-1 (GPC1), Fibronectin (FN), Prostate-specific membrane antigen (PSMA), and functional nucleic acids, such as microRNA-145 have clinical implication for early cancer diagnostics. Moreover, exosomes are widely present in biofluids such as serum, urine, amniotic fluid, cerebrospinal fluid, saliva, and even tears; and hence provide a non-invasive unique feature for cancer diagnosis. Therefore, exosomes have attracted increasing attention for cancer diagnostics, monitoring and prognosis in liquid biopsy. Reliable methods and tools for isolation, quantification, and characterization of cancer exosomes are crucial to propel the development in this field.

The conventional methods for isolation of exosomes include ultracentrifugation (UC), filtration, and density gradient separation, etc. Among them, UC has been considered as the “gold standard” for exosome isolation. However, these conventional isolation methods are mechanically based and are time-consuming. Also, these methods lack the specificity to differentiate the tumorigenic and non-tumorigenic exosomes.

Nanoparticle tracking analysis (NTA), transmission electron microscopy (TEM), or flow cytometry is usually used to analyze the exosomes. NTA offers a rough value of vesicles number, but requires the sample at a high concentration level (1×10⁷-10⁹ particles/mL). For early cancer diagnostics in which the exosomes are usually present at a low concentration level, NTA cannot provide an accurate measure of the biomarkers for monitoring the cancer progress. Western blot and ELISA analysis is regarded as “gold standard” method but is still limited by the poor sensitivity as well as the large amounts of samples requirement. Flow cytometry can be used for high throughput sorting of exosomes with fluorescent labels. However, this method is not effective because the exosomes are often bound to beads and weak light scattering of flow cytometry may cause the number loss.

Electrical based methods including electrohydrodynamic systems and electrochemical biosensors, especially aptamer-based electrochemical sensors (aptasensors), have been adopted to detect the exosomes. Electrohydrodynamic system utilizes the surface shear forces to reduce nonspecific adsorption and improve the specificity, but the limit of detection (LOD) is not sufficient for many applications. Aptasensors have the merits of electrochemical detection methods such as rapid, sensitive, low-consumption and continuous monitoring. However, due to the unpredictable secondary structures of the aptamers, appropriate aptamers are still difficult to obtain and efficient aptamer selection methods are yet to be developed. More recently, new techniques such as surface plasmon resonance (SPR) and Raman scattering enable real-time and label-free readout of the target exosomes. Nevertheless, these methods are still challenging for clinical applications from the throughput and cost aspects.

Droplet or microwell based microfluidics has been demonstrated as the “miniaturized reactors” that revolutionize the biological and chemical assays that are performed in traditional pipette, beaker, tube, or flask. Scaling down the reaction volume in small droplets or wells brings various unique features such as high-throughput, minimal reagent consumption, contamination-free analysis, fast response, miniaturized sample loss, and isolation for parallel reaction. With the explosive advancement in the past decade, droplet microfluidics has emerged as a versatile platform for molecule detection, material synthesis, compartmentalized reactions or high throughput screening in the field of chemistry and biology.

### BRIEF SUMMARY OF THE INVENTION

This disclosure provides applications of microfluidic technology for quantification, isolation, and characterization of exosomes. In certain embodiments, exosomes in a sample are quantified by contacting a sample containing a plurality of exosomes with i) a capture bead comprising a bead conjugated to a first binding agent, and ii) a second binding agent comprising a detectable label, wherein the first binding agent specifically binds to a first biomolecule present in the plurality of exosomes to produce a first complex comprising the capture bead and a first exosome, the second binding agent specifically binds to a second biomolecule present in the plurality of exosomes to produce either an exosome-second binding agent complex comprising the second binding agent and a second exosome or a second complex comprising the capture bead, the first exosome, and the second binding agent; b) from the composition produced at the end of step a), separating the capture beads, the first complexes, and the second complexes, c) from the composition produced at the end of step b), separating from each other each the capture beads, the first complexes, and the second complexes, d) optionally, contacting the separated capture beads, the first complexes, and the second complexes with a substrate that produces a detectable signal from the second binding agent present in the second complexes, and e) detecting the detectable signal from the second complexes to quantify the exosomes in the sample. The relative proportion of beads in the second complexes compared to the capture beads and the first complexes can be used to quantify exosomes in the sample.

The first binding agent and the second binding agent can bind to one or more cancer biomarkers. Thus, the methods disclosed herein can be used to isolate exosomes that are indicative of a cancer. Accordingly, certain embodiments of the invention provide a method for diagnosing a cancer by quantifying in a sample obtained from a subject the exosomes containing cancer biomarkers.

### BRIEF DESCRIPTION OF THE DRAWINGS

FIG. 1 shows exemplary procedures of preparing exosome immunocomplex on beads.

FIG. 2 shows a schematic of digital quantification of the exosomes with specific proteins using droplet or well based methods.

FIG. 3 shows a schematic of the isolation of the desired exosomes with specific biomarkers using droplet sorting.

FIG. 4 shows a schematic of single exosome assay platform with using droplet fusion and sorting technology.

FIGS. 5a to 5d show schematic showing the droplet digital ExoELISA for exosome quantification. (FIG. 5a) Single exosome immunocomplex constructed on a magnetic bead. (FIG. 5b) Substrate and beads are co-encapsulated into microdroplets. (FIG. 5c) Droplet digital ExoELISA chip. (FIG. 5d) Fluorescent readout for counting the positive droplets with the target exosomes.

FIGS. 6a to 6c show characterization of exosomes. (FIG. 6a) TEM shows exosomes with double-wall lipid membrane layers ranging approximately 30-150 nm in diameter. (FIG. 6b) Size distribution of MDA7 MB-231 exosomes by NTA analysis. The band depicts three repetitive experiments. (FIG. 6c) The expression of CD63 (the exosomal marker) and GPC-1 (the diagnostic marker) in MDA-MB-231 exosomes and parent cells by western blot analysis. Equal amounts of proteins (20 μg) in exosomes and cells were loaded.

FIGS. 7a to 7h show droplet generation. (FIG. 7a) Prepared beads and FDG substrate are co-encapsulated into 40 μm diameter droplets which spread in one layer in the device for detection. (FIG. 7b) Droplet digital ExoELISA calibration results showing the dynamic range of the captured exosomes spans 5 orders of magnitude. Dashed line is the background plus 3 times of standard deviation indicating the LOD (˜10 exosomes/μL). (FIG. 7c) Negative control without target exosomes. (FIGS. 7d-7h) gradient of the fluorescence readout by serial dilution of the exosome sample isolated from MDA-MB-231. NanoSight was used as a benchmark measurement for the exosome number concentration.

FIGS. 8a to 8b show specificity of the assay. Specificity of the assay. (FIG. 8a) Western blot analysis showing different expressions of GPC-1 in MDA-MB-231 cells (positive control) and exosomes isolated from MDA-MB-231, HL-7702, RAW264.7, and hES cell culture media. Each lane was loaded with 20 μg proteins. (FIG. 8b) The specificity of the droplet digital ExoELISA with exosomes isolated from MDA-MB-231, HL-7702, RAW264.7, and hES cell culture media. Cases of the magnetic beads without CD63 Ab and detection sample solution without exosomes served as the negative controls. Each sample solution contained 6.39×104 8 exosome particles per μL.

FIGS. 9a to 9c show clinical analyses of GPC-1(+) exosomes by droplet digital ExoELISA. (FIG. 9a) Quantification of GPC-1(+) exosomes from serum samples of 5 healthy samples (HS), 5 patients with benign breast disease (BBD), 12 patients with breast cancer (BC). (FIG. 9b) Scattered dot plots show significant overexpression of GPC-1(+) exosomes of BC patients compared to HS and BBD (****, p<0.0001). (FIG. 9c) Quantification of GPC-1(+) exosomes in 2 patients with breast cancer (BC) and breast cancer after surgery (BC-AS). Error bars represent the standard deviation of three independent experiments.

FIGS. 10a to 10f show dual-color super-resolution images of CD63 and GPC-1 in exosomes isolated from MDA-MB-231 cell culture media. Stochastic optical reconstruction microscopy (STORM) images showing (FIG. 10a) exosome membrane stained with PKH67; (FIG. 10b) CD63 labelled with Alexa Fluor 647; (FIG. 10c) merged image of (FIG. 10a) and (FIG. 10b); (FIG. 10d) exosome membrane stained with PKH67; (FIG. 10e) GPC-1 labelled with Alexa Fluor 647; (FIG. 10f) merged image of (FIG. 10d) and (FIG. 10e).

FIGS. 11a to 11b show TEM images showing an immunomagnetic captured single exosome. (FIG. 11a) PBS was used instead of MDA-MB-231 exosome solution as a negative control. (FIG. 11b) An MDA-MB-231 exosome was captured on a CD63 antibody-conjugated bead. The arrow indicates a single exosome.

FIGS. 12a to 12b show bright field images captured under the microscope with a 20× objective showing the magnet beads are well separated into droplets. The circles indicate the areas where beads are located in the droplets. (FIG. 12a) when the mean number of beads per droplet was set as ˜0.1, in the field of view, all droplets contained either 0 or 1 bead. (FIG. 12b) when the mean number of beads per droplet was set as ˜0.3, in the field of view, only 1 out 85 droplets contained 2 magnetic beads, 5 out 85 droplets contained 1 magnetic bead, and the rest were empty, which agreed with the Poisson statistics pretty well.

FIG. 13 shows optimization of the incubation time for the FDG catalysis reaction in microdroplets. F and FO are the average fluorescence intensity of signals from all microdroplets and background, respectively. The normalized signal reaches the highest at 30 min. Error bars are the standard deviations of three experiments.

FIGS. 14a to 14c show representative NTA plots showing size distribution of exosomes isolated from (FIG. 14a) HL-7702, (FIG. 14b) RAW264.7, and (FIG. 14c) hES cell culture media. respectively. The band depicts three experiments.

### DETAILED DISCLOSURE OF THE INVENTION

As used herein, the singular forms “a,” “an,” and “the” are intended to include the plural forms as well, unless the context clearly indicates otherwise. Further, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” “comprise,” include the phrases “consisting essentially of,” “consists essentially of,” “consisting,” and “consists.”

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which will depend in part on how the value is measured or determined, i.e., the limitations of the measurement system. Where particular values are described in the application and claims, unless otherwise stated the term “about” meaning within an acceptable error range for the particular value should be assumed.

In the present disclosure, ranges are stated in shorthand, to avoid having to set out at length and describe each and every value within the range. Any appropriate value within the range can be selected, where appropriate, as the upper value, lower value, or the terminus of the range. For example, a range of 1-10 represents the terminal values of 1 and 10, as well as the intermediate values of 2, 3, 4, 5, 6, 7, 8, 9, and all intermediate ranges encompassed within 1-10, such as 2-5, 2-8, and 7-10. Also, when ranges are used herein, combinations and sub-combinations of ranges (e.g., subranges within the disclosed range) and specific embodiments therein are intended to be explicitly included.

The disclosure provides microfluidic approaches for quantification, isolation, and characterization of exosomes. The microfluidic approach include droplet or microwell microfluidic techniques, such as compartmentalization, separation, and sorting. For digital quantification and isolation of the desired exosomes, enzyme-linked immunosorbent assay can be utilized to identify the exosomes containing specific biomarkers. For example, through specific antigen-antibody bindings, the target exosomes are recognized and immobilized onto the capture beads, forming enzyme-linked immunocomplex. The immunocomplex solution is partitioned into a sufficient number of uniform isolated compartments (e.g., microdroplets or microwells) such that each compartment contains one or no beads. When necessary, a substrate is added into each compartment for generating a color or fluorescent or a detectable signal from the beads. For those compartments that contain the beads, the linked enzyme triggers the substrate within the compartments to produce absorbance or fluorescence or electrochemical signal (e.g., current), which is measured to determine the presence and quantity of the exosome immunocomplex. Due to the random nature of the bead preparation and partitioning, both the percentage of beads that contain an immunocomplex and the percentage of partitions that contain a bead follow Poisson distribution. Based on the dependent Poisson statistics of the partitions, the target exosome can be quantified up to a single copy precision. After the target exosomes are recognized by the detectable signal, the partitions (microdroplets or microwells) can be further analyzed using droplet sorting technology (e.g., combined with flow cytometry) or imaging using a camera (for microwell based method). The target exosomes can be retrieved for further analysis of the proteins, nuclear acids presented either on the exosome membranes or within the exosomes.

According, certain embodiments of the invention provide a method for isolation or quantification of exosomes in a sample, comprising the steps of:

a) contacting a sample containing a plurality of exosomes with:
i) a capture bead comprising a bead conjugated to a first binding agent, and
ii) a second binding agent comprising a detectable label,

wherein the first binding agent specifically binds to a first biomolecule present in the plurality of exosomes to produce a first complex comprising the capture bead and a first exosome, the second binding agent specifically binds to a second biomolecule present in the plurality of exosomes to produce either an exosome-second binding agent complex comprising the second binding agent and a second exosome, or a second complex comprising the capture bead, the first exosome, and the second binding agent;

b) from the composition produced at the end of step a), separating the capture beads, the first complexes, and the second complexes,

c) from the composition produced at the end of step b), separating from each other each of the capture beads, the first complexes, and the second complexes,

d) optionally, contacting the separated capture beads, the first complexes, and the second complexes with a substrate that produces a detectable signal from the second binding agent present in the second complexes,

e) detecting the detectable signal from the second complexes.

The steps a) to e) listed above are used throughout this disclosure to refer to the specific steps of the methods of the invention. Also, step d) as listed above, can be performed before step c), but it is preferred to perform step d) after step c).

A skilled artisan can recognize that the steps i) and ii) of contacting a sample with a capture bead and a second binding agent can be performed simultaneously or subsequently with each other. For example, a sample, a capture bead, and a second binding agent can be mixed together. Alternatively, a sample and a second binding agent can be mixed first, followed by adding a capture bead. Moreover, a sample and a capture bead can be mixed first followed by adding a second binding agent. Regardless of the sequence of contacting different components, this steps typically results in the formation of a mixture of the following: capture beads, first complexes, second complexes, and exosome-second binding agent complexes.

If the capture beads and the second binding agent are contacted with a sample subsequent to each other, a washing step can be performed between the two contacting steps. For example, a mixture comprising capture beads, the first complexes, exosomes, and other components of the sample can be washed to remove unbound exosomes and/or other components in the sample. Such washing separates the capture beads and the first complexes, which can then be contacted with a second binding agent comprising a detectable label.

The step of contacting a sample with a capture bead and/or a second binding agent is performed under suitable conditions for appropriate period of time to allow the production of the corresponding binding complexes. Typically, a substantial portion of exosomes containing the appropriate biomolecules present in a sample, for example, more than about 90% of the relevant exosomes present in a sample, bind to the capture beads and/or the second binding agents. A person of ordinary skill in the art can implement appropriate conditions for maximum binding between the binding partners.

The beads used in the instant invention can range in a size from about 0.5 microns to about 20 microns, preferably, from about 1 to 15 microns, more preferably, about 2 to 10 microns, even more preferably, about 3 to 6 microns, and most preferably about 4 to 5 microns. The beads are typically made from inert material, such as agarose or inert polymers. The beads can also be superparamagnetic, i.e., they exhibit magnetic properties in a magnetic field with no residual magnetism once removed from the magnetic field. Exemplary superparamagnetic material includes ferrite or magnetite (Fe₃O₄). Additional superparamagnetic materials suitable for use in the beads are known to a skilled artisan and such embodiments are within the purview of the invention.

The beads can also have a core of a superparamagnetic material covered with an inert material, such as a polymer. Exemplary polymers include polystyrene. Additional materials suitable for producing capture beads are known to a skilled artisan and such embodiments are within the purview of the invention.

Beads are conjugated to a first binding agent to produce capture beads. The first binding agent specifically binds to a first biomolecule present in the exosomes.

For the purposes of the invention the phrase “specific binding” or grammatical variations thereof refer to the ability of a binding agent to exclusively bind to its binding partner while having relatively little non-specific affinity with other biomolecules. Specificity can be relatively determined by binding or competitive binding assays. Specificity can be mathematically calculated by, e.g., about 10:1, about 20:1, about 50:1, about 100:1, 10.000:1 or greater ratio of affinity/avidity in binding to the binding partners versus nonspecific binding to other irrelevant biomolecules. For example, an antibody specifically binding to an antigen has the equilibrium dissociation constant (K_{D}) of lower than about 10⁻⁶ M, lower than about 10⁻⁹ M, or lower than about 10⁻¹² M for the binding between the antibody and the corresponding antigen.

On the other hand, “non-specific binding” refers to the binding that is not based on specific interactions between a binding agent and its binding partner. Non-specific binding may result from non-specific interactions, such as, Van Der Waals forces. For example, K_{D} for the binding between the antibody and a non-specific antigen is typically higher than about 10⁻⁶ M, higher than about 10⁻⁴ M or higher than about 10⁻² M.

The first binding agent can be an antibody, an antigen binding fragment of an antibody, an aptamer, a protein binding partner, or a nucleic acid binding partner of a first biomolecule present in the exosomes. In preferred embodiments, a first binding agent binds to a first biomolecule present in exosomes that is a biomarker for a cancer. Certain such biomolecules include CD9, CD63, CD81, GPC1, FN, PSMA, or microRNA-145. Accordingly, a first binding agent can specifically bind to CD9, CD63, CD81, GPC1, FN, PSMA, or microRNA-145. Additional examples of biomolecules that are biomarkers for a cancer that are present in exosomes are known in the art and such embodiments are within the purview of the invention.

The second binding agent specifically binds to a second biomolecule present in the exosomes. The first binding agent and the second binding agent can bind to the same biomolecule or a different biomolecule. If the first binding agent and the second binding agent bind to the same biomolecule, it is preferable that they bind to different binding sites on the same biomarker. Typically, the second biomolecule is different from the first biomolecule. Thus, the second binding agent specifically binds to a second biomolecule that is different from the first biomolecule to which the first binding agent binds.

For the purposes of the invention, the phrase “a biomolecule present in exosomes” indicates that the biomolecule may be present on the surface of the exosome or in the lumen of the exosomes. Preferably, a biomolecule is present on the surface of the exosome to provide easier access to the biomolecule for a binding agent.

The second binding agent can be an antibody, an antigen binding fragment of an antibody, an aptamer, a protein binding partner, or a nucleic acid binding partner of a second biomolecule present in the exosomes. In preferred embodiments, a second binding agent binds to a second biomolecule present in exosomes that is a biomarker for a cancer. Certain such biomolecules include CD9, CD63, CD81, GPC1, FN, PSMA, or microRNA-145. Accordingly, in certain embodiments, a second binding agent binds to CD9, CD63, CD81, GPC1, FN, PSMA, or microRNA-145. Additional examples of biomolecules that are biomarkers for a cancer and that are present in exosomes are known in the art and such embodiments are within the purview of the invention. For example, Li et al. (2017), Mol Cancer; 16: 145, and Nedaeinia et al. (2017), Cancer Gene Therapy; 24:48-56, describe certain such exosomal biomarkers. Each of the Li et al. and Nedaeinia el al. references is incorporated herein by reference in its entirety.

The capture beads, the first complexes, and the second complexes are separated from the composition produced at the end of step a). In certain embodiments, the beads can be washed with a suitable buffer to remove the exosome-second binding agent complexes and other ingredients that may come from the sample and other reagents.

Washing the beads can be performed by methods known in the art and appropriate for specific beads. For example, beads can be centrifuged after repeated washing to separate the beads from the rest of the components. If the beads are magnetic or superparamagnetic, the beads can be captured using a magnetic field and the rest of the ingredients can be washed with an appropriate buffer. A person of ordinary skill in the art can design appropriate washing methods to separate the capture beads, the first complexes, and the second complexes from the composition produced at the end of step a).

After step b), each of the capture beads, the first complexes, and the second complexes are separated from each other. Thus, the composition produced at the end of step b) is separated into multiple compartments, each compartment containing no bead, one capture bead, one first complex, or one second complex.

In certain embodiments, the step of separating the capture beads, the first complexes, and the second complexes is performed using a droplet generation. In droplet generation, the composition comprising capture beads, first complexes, second complexes (the composition produced at the end of step b)) is divided into droplets, wherein each droplet encapsulates one capture bead, one first complex, or one second complex. For the methods disclosed herein to function as intended, less than about 5%, preferably, less than about 4%, more preferably, less than about 3%, even more preferably, less than about 2%, and most preferably, less than about 1% of the compartments contain two or more beads. Ideally, none of the compartments contains two or more beads.

In exemplary embodiments, droplet generation is performed using two immiscible phases; a continuous phase (composition which is divided into droplets) and a dispersed phase (the phase that forms the droplets). The size of the droplets can be controlled by modulating various parameters, such as the flow rate ratio of the continuous phase and the dispersed phase, interfacial tension between two phases, and the geometry of the channels used for droplet generation.

Droplet generation can be active or passive. In active droplet generation an external energy input, such as electric, magnetic, centrifugal energy, is provided droplet manipulation. Passive droplet generation can be performed using certain microfluidic geometries, namely, cross-flowing, flow focusing, and co-flowing.

Cross-flowing involves a continuous phase and a dispersed phase running at an angle to each other. Typically, these phases run perpendicular to each other, i.e., in a T-shaped junction, with the dispersed phase intersecting the continuous phase. Other configurations such as a Y-junction can also be performed. Dispersed phase extends into the continuous phase and is stretched until shear forces break off a droplet. In a T-junction, flow rate ratio and capillary number control droplet size and formation rate. The capillary number depends on aspects such as the viscosity of the continuous phase, the superficial velocity of the continuous phase, and the interfacial tension. Additional details about cross-flowing droplet generation are well known to a person of ordinary skill in the art and such embodiments are within the purview of the invention.

Flow focusing involves the dispersed phase flowing to meet the continuous phase typically at an angle (nonparallel streams). The dispersed phase then undergoes a constraint that creates a droplet. The constraint is typically a narrow channel, which creates the droplet though symmetric shearing. Slower the flow rate, bigger is the droplet size, and vice versa. Additional details about flow focusing droplet generation are well known to a person of ordinary skill in the art and such embodiments are within the purview of the invention.

In co-flowing the dispersed phase channel is enclosed inside a continuous phase channel and at the end of the dispersed phase channel, the fluid is stretched until it breaks to form droplets either by dripping or jetting. Dripping occurs when capillary forces dominate the system and droplets are created at the channel endpoint and jetting occurs by widening or stretching when the continuous phase is moving slower, creating a stream from the dispersed phase channel opening. In the widening format, the dispersed phase moves faster than the continuous phase causing a deceleration of the dispersed phase, widening the droplet and increasing the diameter. In the stretching format, viscous drag dominates causing the stream to narrow creating a smaller droplet. The droplet size depends on the phase flow rate and on the stretching or widening format. Additional details about co-flowing droplet generation are well known to a person of ordinary skill in the art and such embodiments are within the purview of the invention.

Typically, a composition produced at the end of step b) is used as the droplet phase and a continuous phase is provided, for example, containing an oil or emulsion. Particular details about the droplet generation step depend on the intended size of the droplet, the type of sample tested, the content of biomarkers in the exosomes, etc., and a person of ordinary skill in the art can determine such conditions as needed and such embodiments are within the purview of the invention. Certain such embodiments are described in the Examples 1-4 below.

As noted above, the composition produced at the end of step b) is separated into multiple compartments, each compartment containing no bead, one capture bead, one first complex, or one second complex. In certain embodiments, the step of separating the capture beads, the first complexes, and the second complexes is performed using microwells. For example, the composition produced at the end of step b) can be introduced onto a support comprising microwells.

A “microwell” refers to a well having a volume of between 1 fl to 1000 nl, preferably, between 50 nl to 900 nl, more preferably, between 150 nl to 700 nl, even more preferably, between 250 nl to 600 nl, and most preferably, about 500 nl. The size of the microwells on a chip is such that only one capture bead, only one first complex, or only one second complex would fit into one microwell. Therefore, the size of a microwell can be selected based on the size of capture beads.

One example of a support comprising microwells is a glass bottom bonded to a silicon grid that creates the microwells. A support comprising microwells can also be made from poly(dimethylsiloxane) polymer or plastic. Additional materials suitable for preparing a support comprising microwells are known to a skilled artisan and such embodiments are within the purview of the invention.

Once the capture beads, the first complexes, and the second complexes are separated from each other, the number and/or the amount of the second complexes can be determined based on the detectable signal provided by the second binding agent.

In the methods of the invention, one capture bead can contain thousands of molecules of first binding agent that are able to capture the exosome. By controlling the ratio of beads to exosomes, one can ensure that one capture bead binds to no more than one exosome. Each exosome can then bind to one or more molecules of the second binding agent. E.g., one capture bead can bind to one exosomes and each exosome can bind to several molecules of the second binding agent. Hence, more molecules of the second binding agent would give a relatively stronger signal. Therefore, quantification of exosomes in a sample can be performed based on the number of capture beads and the intensity of the signal produced by each of the capture beads.

As noted above, the second binding agent contains a detectable label. Therefore, the second complex can be distinguished from the capture beads and the first complexes based on the presence or absence of the detectable signal.

The detectable label can produce a detectable signal with or without a substrate. For example, if the detectable label is a fluorescent, radioactive, or chemiluminescent molecule, the second binding agent can produce a detectable signal without a substrate. On the other hand, if the detectable label is an enzyme that acts on a substrate to produce a detectable signal, a substrate is provided to produce the detectable signal, which is then detected to detect the second complex.

Detectable labels suitable for use in the methods disclosed herein include, but are not limited to, fluorescent moieties, chemiluminescent and bioluminescent reagents, enzymes, and radioisotopes. Fluorescent moieties include, but are not limited to, fluorescein, fluorescein isothiocyanate, Cascade Blue, rhodamine, dichlorotriazinylamine fluorescein, dansyl chloride, Texas Red, Oregon Green, cyanines (e.g., CY2, CY3, and CY5), umbelliferone, allophycocyanine or phycoerythrin. An example of a luminescent material includes luminol. Examples of bioluminescent materials include, but are not limited to, luciferin, green fluorescent protein (GFP), enhanced GFP, and aequorin. Enzymes that can be used include but are not limited to luciferase, beta-galactosidase, acetylcholinesterase, horseradish peroxidase, glucose-6-phosphate dehydrogenase, and alkaline phosphatase.

When the detectable label is an enzyme, a suitable substrate is provided to the enzyme for production of a detectable signal. For example, if the detectable label is a peroxidase, the substrate can be hydrogen peroxide (H₂O₂) and 3-3′ diaminobenzidine or 4-chloro-1-naphthol. Other substrates suitable for use with other enzymes are well known in the art.

Isotopes that can be used include, but are not limited to, ¹²⁵I, ¹⁴C, ³⁵S, and ³H.

If a second binding agent requires a substrate for producing a detectable signal, the separated capture beads containing the first binding agent, the first complexes, and the second complexes are contacted with the substrate that produces a detectable signal from the second binding agent. The step of contacting a substrate to the separated beads can be performed in various ways depending on the method used to separate the beads.

For example, if a support comprising microwells is used to separate the beads, a substrate is introduced into the microwells and incubated under appropriate conditions for an appropriate period of time for the production of a detectable signal. The substrate can be introduced in the form of a suitable composition, for example, a buffer. Depending upon the type of the enzyme used as a detectable label, the excess substrate can be washed before detecting the signal.

If droplet generation is performed to separate the beads, a substrate can be incorporated in the continuous or the droplet phase. (FIG. 4.)

If a second binding agent does not require a substrate for producing a detectable signal, the separated capture beads, the first complexes, and the second complexes are tested for the detectable signal to identify and quantify the second complexes. The step of detecting the signal depends on the type of signal to be detected. For example, if a detectable signal is a fluorescent emission, fluorescent camera can be used. Additional methods of detecting specific detection signals are well known in the art and can be readily identified by a person of ordinary skill in the art. Such embodiments are within the purview of the invention.

Detecting the signal from the second complexes can be used to distinguish the second complexes from the capture beads containing the first binding agent and the first complexes. Such detection can be performed in various ways depending upon the method used to separate the beads.

For example, if a support comprising microwells is used for separating the beads, a camera can be used to image the microwells and identify the number of microwells containing the capture beads, and the first complexes, and the second complexes. If droplet generation is used for separating the beads, flow cytometry can be performed used to identify the number of droplets containing the capture beads, and the first complexes, and the second complexes.

The relative number of second complexes compared to the capture beads and the first complexes as well as the intensity of the detectable signal from each of the second complexes can be used to quantify the second complexes, and thereby, the exosomes in the sample. A standard curve can be used with control samples containing known amounts of exosomes to further facilitate quantification of exosomes in a sample. A skilled artisan can design appropriate standard curve for such quantification and such embodiments are within the purview of the invention.

Exosomes can be used as biomarkers for cancer diagnostics. Exosomes shed from tumor tissues and carry numerous cancer biomarkers such as transmembrane and cytosolic proteins (CD9, CD63, CD81, etc.), lipids, DNA and microRNA. Special proteins such as GPC1, FN, PSMA and functional nucleic acids such as microRNA-145 can be used for early cancer diagnostics. Moreover, exosomes are widely present in human biofluids such as serum, urine, amniotic fluid, cerebrospinal fluid, saliva, and even tears; and hence provide a non-invasive unique feature for cancer diagnosis. Therefore, detecting and quantifying exosomes according to the methods described herein can be used for cancer diagnostics, monitoring, and prognosis.

Accordingly, certain embodiments of the invention provide a method of detecting a cancer in a subject, the method comprising:

(I) determining the level of exosomes containing one or more cancer biomarkers in:
i) a test sample obtained from the subject, and
ii) optionally, a control sample;

(II) optionally obtaining a reference value corresponding to the level of exosomes containing one or more cancer biomarkers,

(III) identifying the subject as:
i) having the cancer based on the level of exosomes containing one or more cancer biomarkers in the test sample compared to the level in the control sample or the reference value, or
ii) not having the cancer based on the level of exosomes containing one or more cancer biomarkers in the test sample compared to the level in the control sample or the reference value.

If the subject is identified as having a cancer, the method can further comprise administering a therapy to the subject to treat and/or manage the cancer. If the subject is identified as not having a cancer, the method can further comprise withholding the therapy to the subject to treat and/or manage the cancer.

A cancer therapy can be selected from radiotherapy, chemotherapy, surgery, immunotherapy, such as monoclonal antibody therapy (e.g., bevacizumab or cetuximab), or any combination thereof. A therapy administered to a subject depends on the type of cancer, age of a subject, the stage of cancer, and other such individualized parameters.

In preferred embodiments, the methods disclosed above to quantify exosomes in a sample are used to determine the level of exosomes containing one or more cancer biomarkers in a test sample obtained from the subject, and optionally, a control sample. Thus, certain embodiments of the invention provide a method for determining the level of exosomes containing one or more cancer biomarkers in a sample, comprising the steps of:

a) contacting the sample with:
i) a capture bead comprising a bead conjugated to a first binding agent, and
ii) a second binding agent comprising a detectable label,

wherein the first binding agent specifically binds to a first cancer biomarker present in the exosomes to produce a first complex comprising the capture bead and a first exosome, the second binding agent specifically binds to a second cancer biomarker present in the exosomes to produce either an exosome-second binding agent complex comprising the second binding agent and a second exosome or a second complex comprising the capture bead, the first exosome, and the second binding agent;

b) from the composition produced at the end of step a), separating the capture beads, the first complexes, and the second complexes,

c) from the composition produced at the end of step b), separating from each other each of the capture beads, the first complexes, and the second complexes,

d) optionally, contacting the separated capture beads, the first complexes, and the second complexes with a substrate that produces a detectable signal from the second binding agent present in the second complexes,

e) detecting the detectable signal from the second complexes to quantify the exosomes in the sample.

The first binding agent and the second binding agent can be, independently of each other, an antibody, an antigen binding fragment of an antibody, an aptamer, a protein binding partner, or a nucleic acid binding partner of a first cancer biomarker present in the exosomes. Certain such cancer biomarkers include CD9, CD63, CD81, GPC1, FN, PSMA, or microRNA-145. Accordingly, in certain embodiments, a first binding agent binds to CD9, CD63, CD81, GPC1, FN, PSMA, or microRNA-145. Additional examples of cancer biomarkers that are present in exosomes are known in the art and such embodiments are within the purview of the invention.

The first binding agent and the second binding agent can bind to the same cancer biomarker or a different cancer biomarker. If the first binding agent and the second binding agent bind to the same cancer biomarker, it is preferable that they bind to different binding sites on the same cancer biomarker.

The details of the methods discussed above for quantification of exosomes in a sample are also applicable to the diagnostic methods for cancer described herein. For example, the specific binding agents, beads, detectable labels, substrates, methods used for separation of beads, methods used for detection of the detectable signal, methods used for quantification of second complexes, etc., discussed above are also applicable to the diagnostic methods for cancer and such embodiments are within the purview of the invention.

To practice the methods described herein for identifying a subject as having a cancer, control samples can be obtained from one or more of the following:

a) an individual belonging to the same species as the subject and not having a cancer,

b) an individual belonging to the same species as the subject and known to have a low risk or no risk of developing a cancer, or

c) the subject prior to getting a cancer.

Additional examples of control samples are well known to a person of ordinary skill in the art and such embodiments are within the purview of the current invention.

In certain embodiments, the control sample and the test sample are obtained from the same type of an organ or tissue. Non-limiting examples of the organ or tissue which can be used as samples are placenta, brain, eyes, pineal gland, pituitary gland, thyroid gland, parathyroid glands, thorax, heart, lung, esophagus, thymus gland, pleura, adrenal glands, appendix, gall bladder, urinary bladder, large intestine, small intestine, kidneys, liver, pancreas, spleen, stoma, ovaries, uterus, testis, skin, blood or buffy coat sample of blood. Additional examples of organs and tissues are well known to a person of ordinary skill in the art and such embodiments are within the purview of the invention.

In certain other embodiments, the control sample and the test sample are obtained from the same type of a body fluid. Non-limiting examples of the body fluids which can be used as samples include amniotic fluid, aqueous humor, vitreous humor, bile, blood, cerebrospinal fluid, chyle, endolymph, perilymph, female ejaculate, lymph, mucus (including nasal drainage and phlegm), pericardial fluid, peritoneal fluid, pleural fluid, pus, rheum, saliva, sputum, synovial fluid, vaginal secretion, semen, blood, serum or plasma. Additional examples of body fluids are well known to a person of ordinary skill in the art and such embodiments are within the purview of the invention.

The methods described herein can be used to identify a subject as having a cancer. In certain embodiments, the subject is a mammal. Non-limiting examples of mammals include human, ape, canine, pig, bovine, rodent, or feline.

The methods of diagnosing a cancer can be used to diagnose types of cancer including, but not limited to: Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-related cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone cancer, Bone tumor, Brain stem lioma, Brain tumor, Breast cancer, Brenner tumor, Bronchial tumor, Bronchioloalveolar carcinoma, Brown tumor, Burkitt's lymphoma, Cancer of unknown primary site, Carcinoid Tumor, Carcinoma, Carcinoma in situ, Carcinoma of the penis, Carcinoma of unknown primary site, Carcinosarcoma, Castleman's Disease, Central nervous system embryonal tumor, Cerebellar astrocytoma, Cerebral astrocytoma, Cervical cancer, Cholangiocarcinoma, Chondroma, Chondrosarcoma, Chordoma, Choriocarcinoma, Choroid plexus papilloma, Chronic lymphocytic leukemia, Chronic monocytic leukemia, Chronic myelogenous leukemia, Chronic myeloproliferative disorder, Chronic neutrophilic leukemia, Clear-cell tumor, Colon cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial uterine cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing family of tumor, Ewing family sarcoma, Ewing's sarcoma, Extracranial germ cell tumor, Extragonadal germ cell tumor, Extrahepatic bile duct cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric cancer, Gastric lymphoma, Gastrointestinal cancer, Gastrointestinal carcinoid tumor, Gastrointestinal stromal tumor, Gastrointestinal stromal tumor, Germ cell tumor, Germinoma, Gestational choriocarcinoma, Gestational trophoblastic tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin's lymphoma, Hypopharyngeal cancer, Hypothalamic glioma, Inflammatory breast cancer, Intraocular melanoma, Islet cell carcinoma, Islet cell tumor, Juvenile myelomonocytic leukemia, Sarcoma, Kaposi's sarcoma, Kidney cancer, Klatskin tumor, Krukenberg tumor, Laryngeal cancer, Lentigo maligna melanoma, Leukemia, Leukemia, Lip and oral cavity cancer, Liposarcoma, Lung cancer, Luteoma, Lymphangioma, Lymphangiosarcoma, Lymphoepithelioma, Lymphoid leukemia, Lymphoma, Macroglobulinemia, Malignant fibrous histiocytoma, Malignant fibrous histiocytoma of bone, Malignant glioma, Malignant mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Melanoma, Meningioma, Merkel Cell Carcinoma, Mesothelioma, Metastatic squamous neck cancer with occult primary, Metastatic urothelial carcinoma, Mixed mullerian tumor, Monocytic leukemia, Mouth cancer, Mucinous tumor, Multiple endocrine neoplasia syndrome, Multiple myeloma, Mycosis fungoides, Myelodysplasia disease, Myelodysplasia syndromes, Myeloid leukemia, Myeloid sarcoma, Myeloproliferative disease, Myxoma, nasal cavity cancer, Nasopharyngeal cancer, Nasopharyngeal carcinoma, Neoplasm, Neurinoma, Neuroblastoma, Neuroblastoma, Neurofibroma, Neuroma, Nodular melanoma, Non-Hodgkin's lymphoma, Nonmelanoma skin cancer, Non-small cell lung cancer, Ocular oncology, Oligoastrocytoma, Oligodendroglioma, Oncocytoma, Optic nerve sheath meningioma, Oral cancer, Oropharyngeal cancer, Osteosarcoma, Osteosarcoma, Ovarian cancer, Ovarian epithelial cancer, Ovarian germ cell tumor, Ovarian low malignant potential tumor, Paget's disease of the breast, Pancoast tumor, Pancreatic cancer, Papillary thyroid cancer, Papillomatosis, Paraganglioma, Paranasal sinus cancer, Parathyroid cancer, Penile cancer, Perivascular epithelioid cell tumor, Pharyngeal cancer, Pheochromocytoma, Pineal parenchymal tumor of intermediate differentiation, Pineoblastoma, Pituicytoma, Pituitary adenoma, Pituitary tumor, Plasma cell neoplasm, Pleuropulmonary blastoma, Polyembryoma, precursor T-lymphoblastic lymphoma, Primary central nervous system lymphoma, Primary effusion lymphoma, Primary hepatocellular cancer, Primary liver cancer, Primary peritoneal cancer, Primitive neuroectodermal tumor, Prostate cancer, Pseudomyxoma peritonei, Rectal cancer, Renal cell carcinoma, Respiratory tract carcinoma involving the NUT gene on chromosome 15, Retinoblastoma, Rhabdomyoma, Rhabdomyosarcoma, Richter's transformation, Sacrococcygeal teratoma, Salivary gland cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin cancer, Small blue round cell tumor, Small cell carcinoma, Small cell lung cancer, Small cell lymphoma, Small intestine cancer, Soft tissue sarcoma, Somatostatinoma, Soot wart, Spinal cord tumor, Spinal tumor, Splenic marginal zone lymphoma, Squamous cell carcinoma, Stomach cancer, Superficial spreading melanoma, Supratentorial primitive neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat cancer, Thymic carcinoma, Thymoma, Thyroid cancer, Transitional Cell Cancer of renal pelvis and ureter, Transitional cell carcinoma, Urachal cancer, Urethral cancer, Urogenital neoplasm, Uterine sarcoma, Uveal melanoma, Vaginal cancer, Verner-Morrison syndrome, Verrucous carcinoma, Visual pathway glioma, Vulvar cancer, Waldenstrom's macroglobulinemia, Warthin's tumor, Wilms' tumor, or any combinations thereof. In preferred embodiments, the methods of diagnosing a cancer according to the instant invention can be used to diagnose, brain tumor, breast cancer, gastrointestinal cancer, colorectal cancer, lung cancer, or prostate cancer.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1—Construction of Exosome Immunocomplex on Beads

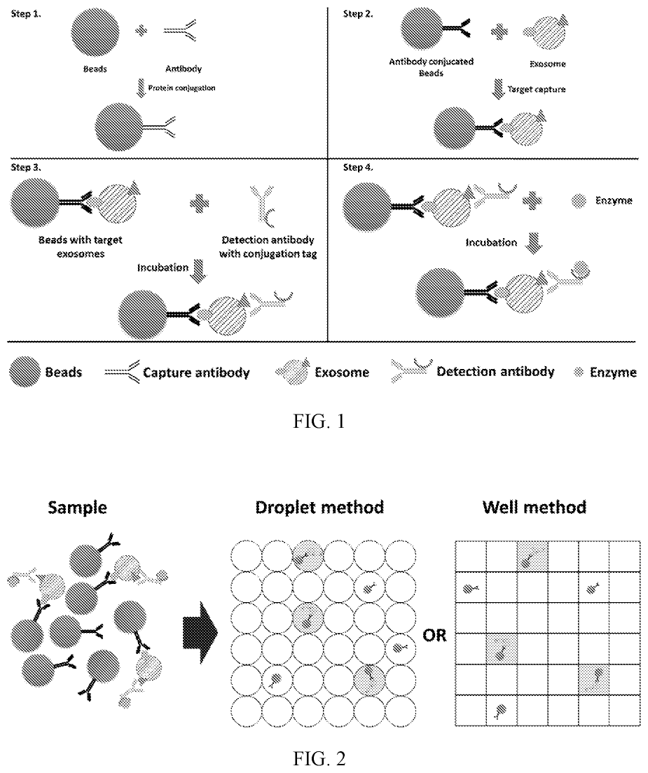

Digital enzyme-linked immunosorbent assays in various microfluidic platforms are demonstrated. Exosome solutions are obtained from biofluids and prepared through ultracentrifugation, ultrafiltration, density-gradient separation, and immunoaffinity capture methods. Since antigens exist on the surface of exosome, they can be recognized by the specific antibodies. One pair of antibodies which identify the exosome is constructed onto the bead in the form of an immunocomplex. The construction of immunocomplex onto the beads is shown in FIG. 1. The antibodies which can recognize the biomarkers (e.g. CD63) on the surface of exosomes are conjugated to the beads (e.g., Dynabeads™ or agarose beads). The beads are then incubated with an exosome solution. After incubation, the beads are collected by magnetic force or centrifugation. After thorough washing, the target exosomes conjugated on the beads are purified from the sample solution. Next, a second antibody which can recognize the same (e.g., CD63) or different biomarkers (e.g., GPC-1) on the exosome is used to detect the exosome. The detection antibody is usually conjugated to a tag (e.g., biotin) which can recognize the enzyme (e.g., streptavidin conjugated beta-galactosidase). The method disclosed herein for quantification and isolation of the exosomes is not limited to a specific biomarker. Different exosome biomarkers that have been discovered on the exosome membranes with corresponding antigen-antibody pairs are applicable.

### Example 2—Digital Quantification of the Target Exosomes

Digital quantification is carried out of the immunocomplex beads bound to the target exosomes via specific protein biomarkers. The immunocomplex constructed beads solution is flown into the channel to mix the solution with another channel of a substrate (e.g., FDG) flow and to form droplets of the mixtures. Instead of using droplets as the compartments, microwells fabricated on a flat chip can also be utilized to compartmentalize the sample solution. The sample with beads can be first dropped on the chip and be scraped into the wells. The substrate (e.g., FDG) solution is added into each compartment subsequently. The microwell chip is then sealed on the top to isolate each individual space for reaction. The microfluidic workflow is schematically shown in FIG. 2. After incubation, the droplets/wells with beads constructed immunocomplex emit color or fluorescent or electrochemical signal for detection. The signal can be detected by fluorescence microscope or electrochemical sensor array. By counting the positive and negative droplets/wells number, the number of the target exosomes can be calculated according to the two dependent Poisson equation:

N=−N_{b}ln[1+vₛ/v_{d}N_{b} ln(1−p)]

Where N is the absolute number of the captured molecules, N_{b} is the total number of beads, Vₛ is the total testing sample volume, V_{d} is the droplet/well volume, and p is the ratio of positive to total droplets/wells number.

### Example 3—Exosome Isolation

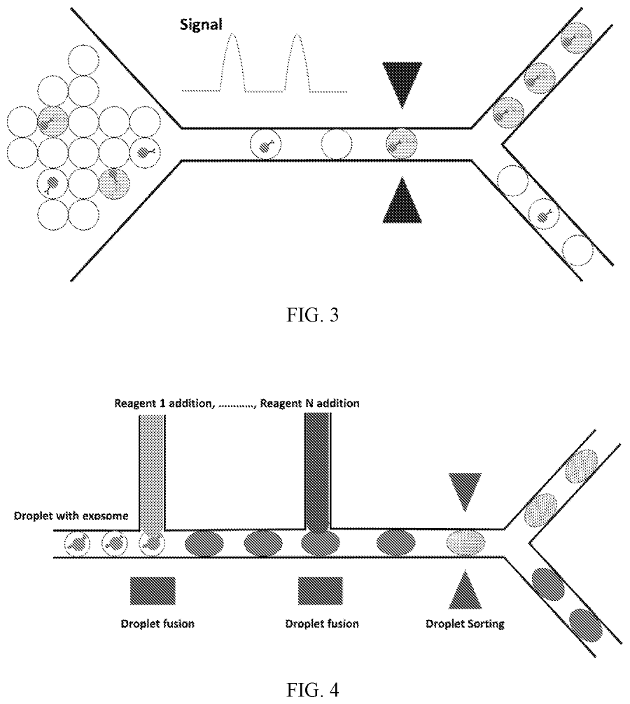

By constructing the immunocomplex on the beads and encapsulating them into droplets, the signal from labelled fluorescein or chemiluminescence can be used as a trigger for droplet sorting. The droplets that contain target exosomes can be separated through droplet sorting technology including electric sorting, mechanical sorting or acoustic sorting. FIG. 3 shows a schematic of the isolation of the fluorescent exosomes with desired information.

### Example 4—Exosome Characterization

Because exosomes shed from tumor tissues carry numerous biomarkers such as proteins, DNA or microRNA, etc., to characterize and analyze the content of each exosome individually, droplet microfluidics can be used for high-throughput assays. FIG. 3 shows a schematic of the characterization of exosomes through droplet fusion, sorting or other droplet manipulation technology. By diluting and encapsulating exosomes into sufficient number of droplets, exosome assays can be performed at the single exosome level. By adding the reagent into the droplets with exosome, the information contained in an individual exosome can be studied. The reagent being added into the droplets can be the exosome lysis buffer, PCR mix, RT mix, etc.

### Example 5—Single-Exosome-Counting Immunoassays for Cancer Diagnostics

Exosomes shed by tumor cells have been recognized as promising biomarkers for cancer diagnostics due to their unique composition and functions. Quantification of low concentrations of specific exosomes present in very small volumes of clinical samples may be used for noninvasive cancer diagnosis and prognosis. An immunosorbent assay is provided for digital quantification of target exosomes using droplet microfluidics. The exosomes were immobilized on magnetic mircobeads through sandwich ELISA complexes tagged with an enzymatic reporter that produces a fluorescent signal. The constructed beads were further isolated and encapsulated into a sufficient number of droplets to ensure only a single bead was encapsulated in a droplet. The droplet-based single-exosome-counting enzyme-linked immunoassay (droplet digital ExoELISA) approach enables absolute counting of cancer-specific exosomes to achieve unprecedented accuracy. A limit of detection (LOD) was achieved down to 10 enzyme-labeled exosome complexes per microliter (˜10^{—17} M). The application of the droplet digital ExoELISA platform in quantitative detection of exosomes in plasma samples directly from breast cancer patients is demonstrated. Early diagnosis of cancer and accelerated discovery of cancer exosomal biomarkers for clinical diagnosis can be achieved using the methods disclosed herein.

Evidence has indicated that the exosome molecular cargo shed from tumor tissues can be identified as potential non-invasive biomarkers for cancer diagnosis because it reflects the genetic or signaling alterations of the parent tumors. For instance, Glypican-1 (GPC-1), an exosomal membrane protein, was discovered to have much higher expression on the cancerous exosomes than the noncancerous by immunoblotting analysis, revealing its clinical value as an exosomal biomarker for the early diagnosis of pancreatic, breast, and colorectal cancer.

Exosomes secreted by nucleated cells are widely present in human bio-fluids and various exosome subpopulations exist. Recently, the subpopulation of tumor-derived exosomes was found to be valuable for clinical diagnostics. To accurately quantify and classify the tumor derived exosomes from bio-fluids is potentially significant for cancer diagnostics, prognosis, and monitoring the response of therapy. Conventional methods such as nanoparticle tracking analysis (NTA), western blot, ELISA, and flow cytometry have been widely adopted in research labs for exosome quantity measurement. However, NTA only provides an estimated number of exosomes at a high concentration level (1×10⁷-10⁹ particles/mL) and lacks specificity. Western blot, ELISA and flow cytometry all require large amounts of sample input and have limited sensitivity. Unfortunately, in the early stage of cancer, limited tumor-derived exosomes in peripheral blood circulation can hardly be detected with these conventional quantification methods. Many efforts have been made by researchers to improve the sensitivity of the detection methods, including miniaturized microfluidic platforms, aptamer-based electrochemical sensors, surface plasmon resonance (SPR), and Raman scattering. However, these detection methods are performed in a bulk solution, which hardly enables absolute quantification or classification. As the cancer biomarkers that present in the early stage in liquid biopsy are at low concentrations in the range of 10⁻¹² to 10⁻¹⁶ M, to quantitate such low abundance markers, the required sensitivity for detection needs to be at the single molecule level. Recently, single extracellular vesicle analysis (SEA), based on photon counting techniques, has been applied for multiplexed profiling of single extracellular vesicles using ELISA. Careful buffer washing and complex imaging procedures are required to differentiate single vesicles from protein complexes or other clusters due to their low signal-to-noise ratios, and the detection limit is still quite high (e.g., with an intensity cutoff of 10² counts). Nevertheless, these methods are still impractical for wide adoption due to the throughput and cost. Reliable platforms for quantification of exosomes with high sensitivity and specificity are still lacking.

In recent years, digital PCR and digital ELISA platforms have revolutionized detection technologies for absolute quantification of nucleic acids and proteins. In contrast to the conventional biological and chemical assays conducted in large volumes, in pipettes, beakers, tubes or flasks, the basic principle of digital quantification of molecules is to divide the sample uniformly into a large quantity of small compartments (either in microwells or in droplets). By doing so, an individual molecule is confined in a small volume where the signal can be amplified and concentrated for detection. Compartmentalization technology that ensures the isolation of molecules in each compartment to follow the Poisson distribution is the core to the success of digital quantification. Droplet microfluidics that generates uniform droplets at the pico- to nanoliter scale in high throughput (in kHz) has enabled numerous single-molecule assays to be performed in parallel. In recent years, there has been tremendous progress in the development of droplet-based platforms for the formation and manipulation of monodispersed droplets and the associated use of a range of fluorescence-based techniques for high-throughput and highly sensitive analysis of droplet content.

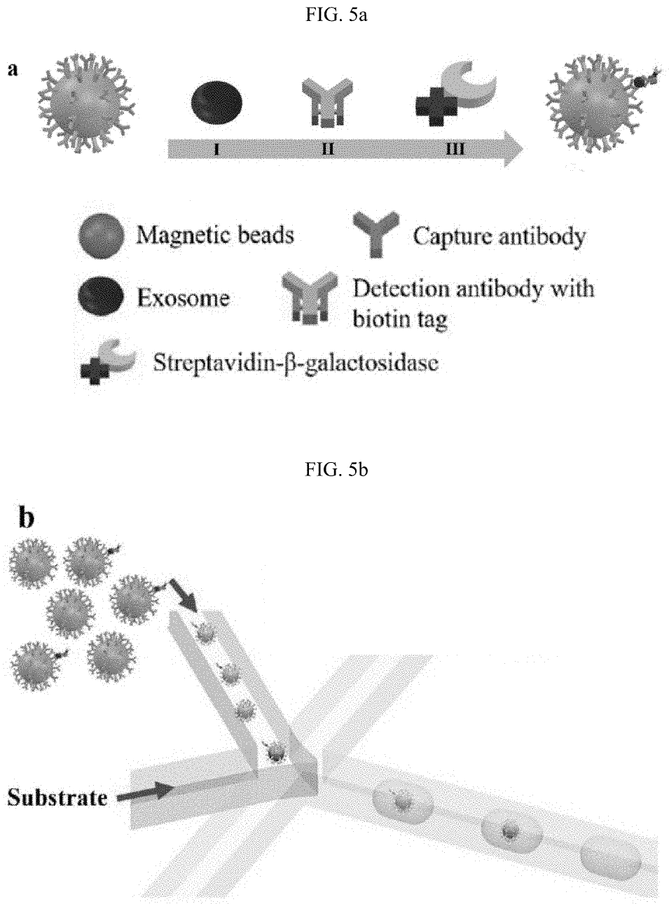

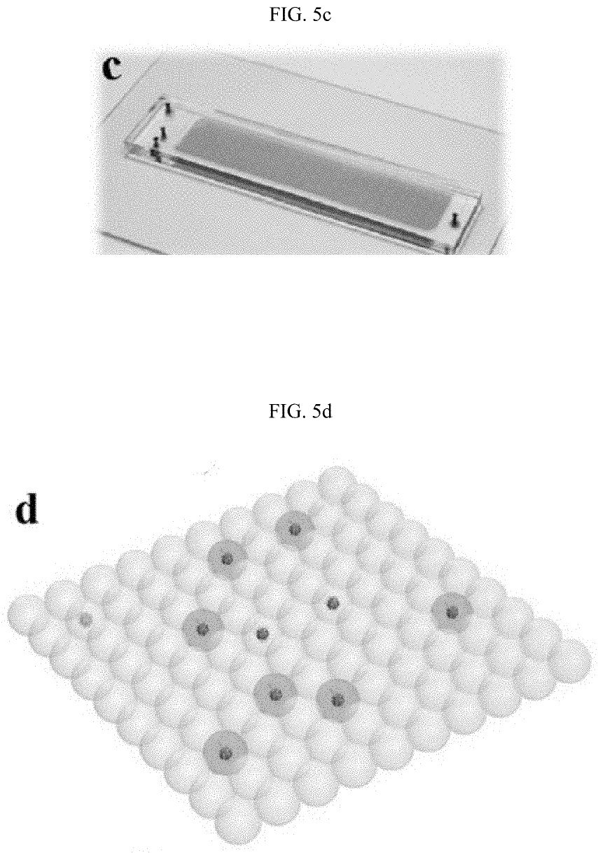

A droplet-based single-exosome-counting immunoassay approach is developed for digital quantification of exosomes. Exosome enzyme-linked immunosorbent assay (ExoELISA) is adopted to identify the exosomes with target membrane protein biomarkers. This method is also herein referenced as droplet digital ExoELISA, the procedure of which is illustrated in FIGS. 5a-5d. Magnetic beads serve as a medium for capture and separation of the target exosomes. First, the exosome suspension is mixed with a sufficient number of magnetic beads conjugated with capture antibodies that can selectively bind a specific protein on the exosome membrane. After effective magnetic separation and washing, one target exosome is immobilized and captured onto a magnetic bead. A detection antibody tagged with an enzymatic reporter further recognizes the antigen on the captured exosome, forming a single enzyme-linked immunocomplex on the bead (FIG. 5a). Second, the prepared beads and the enzymatic substrate are co-encapsulated into a sufficient number of microdroplets to ensure that a majority of droplets contain no more than one bead, using a microfluidic chip (FIGS. 5b-5c). Third, for those droplets that contain the beads with exosome immunocomplex, the substrate is catalyzed by the enzyme to emit fluorescein within the droplets (FIG. 5d). Based on the statistics of the fluorescent droplets, the target exosome concentration can be calculated. The droplet digital ExoELISA approach is able to detect as few as ˜5 exosomes per μL. Other than high sensitivity, the droplet digital ExoELISA offers high specificity and absolute quantification for targeting exosomes with specific protein biomarkers. For clinical demonstration, the GPC-1(+) exosomes from breast cancer patients and the results yielded distinct GPC-1(+) expression level before and after surgery, suggesting the great potential of the droplet digital ExoELISA platform for cancer diagnostics.

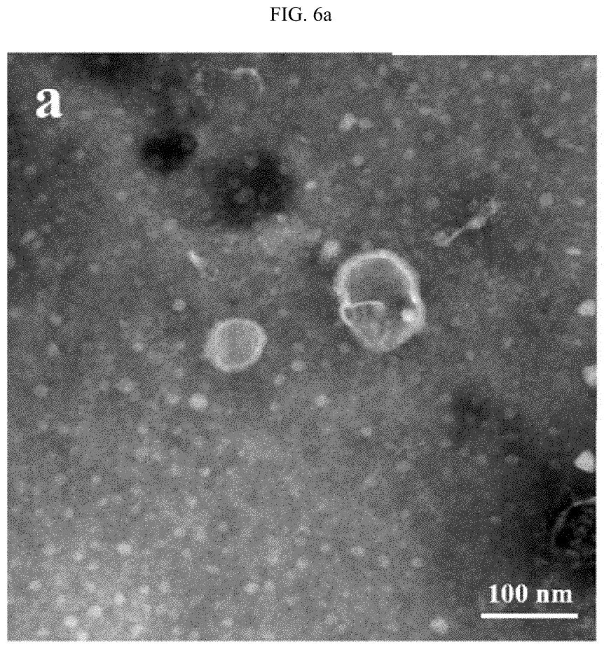

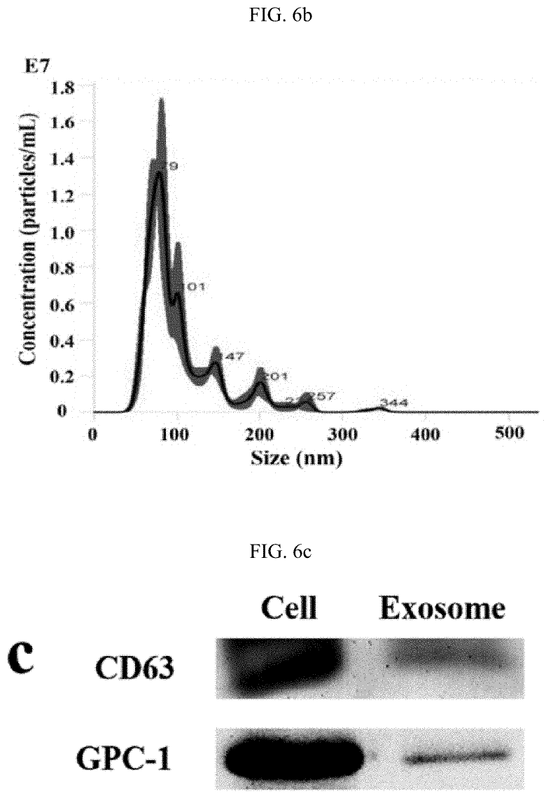

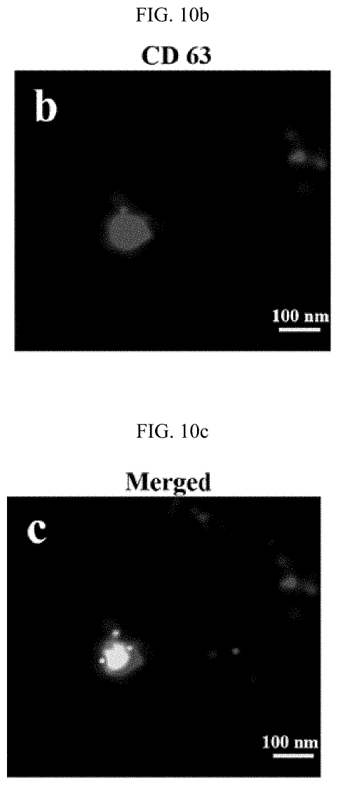

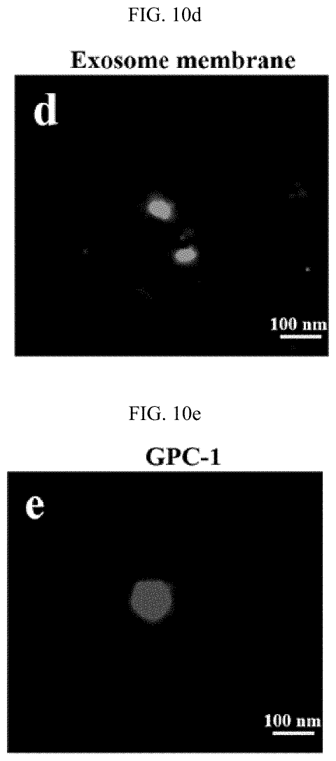

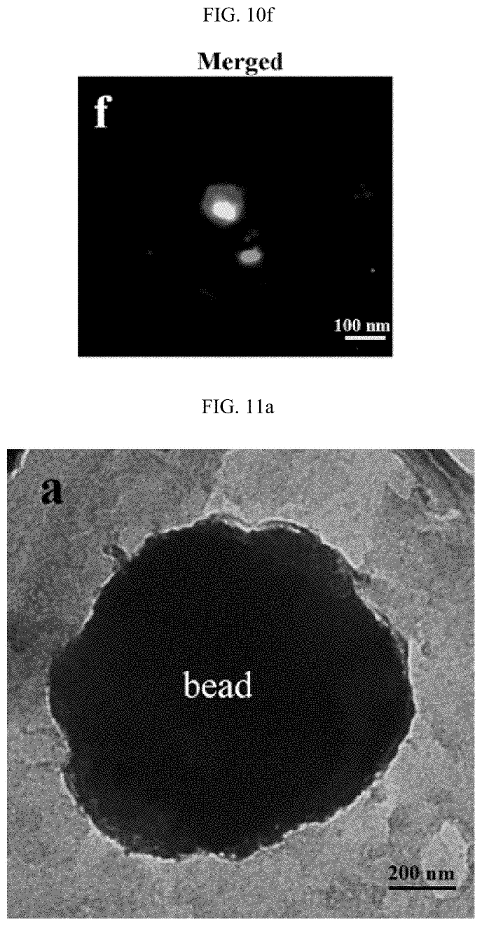

Exosomes were purified and isolated from a breast tumor cell line (MDA-MB-231) by multiple steps of ultracentrifugation following our previous work. Standard characterization of exosomes was performed using transmission electron microscopy (TEM), NTA and western blot, respectively. As shown in FIG. 6a, the TEM image revealed the lipid bilayer structure remained intact on the purified exosomes after ultracentrifugation and the size of the exosomes ranged from 50 nm to 150 nm in diameter. With NTA analysis, the size distribution and concentration of the exosomes was determined (FIG. 6b). The prepared exosomes had an average size of 104.2±3.9 nm in diameter and the corresponding concentration was 6.39×10⁸±4.90×10⁶ particles per mL. CD63 protein, a member of the transmembrane 4 superfamily, was selected as the protein biomarker for capturing exosomes because CD63 is the exosome-enriched protein located on the membrane and, according to the literature, is commonly used for exosome capture. Western blot analysis showed the exosomal marker CD63 on the exosomes isolated from the MDA-MB-231 culture media was consistent with the CD63 protein extracted from the same cell line as a positive control, indicating the existence of CD63 on these samples (FIG. 6c, top row). Also, a dual color super resolution microscopy was used to confirm the localization of CD63 on the exosome membrane (FIGS. 10a-10c). GPC-1 protein was selected as the breast cancer reporter. The high expression of GPC-1 on exosomes from the MDA-MB-231 cell line and the location of GPC-1 on exosome membranes was confirmed by western bolt analysis (FIG. 6c, bottom row) and the dual-color super resolution microscopy (FIGS. 10d-10f). Thus, the isolated breast cancer exosomes can be further used for the construction of exosome immunocomplexes on magnetic beads using ExoELISA.

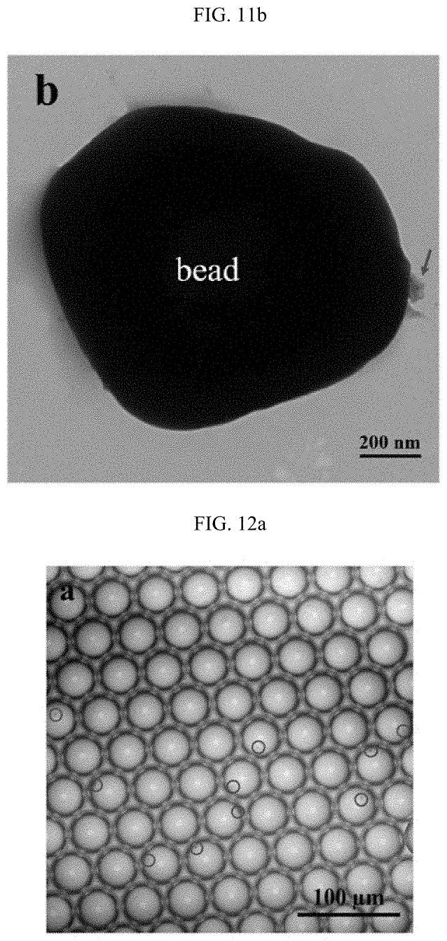

A protocol to construct single exosome immunocomplexes on beads was developed. First magnetic beads conjugated with CD63 antibody were prepared. The functionalized beads were then used for capturing exosomes. The probability of the number of exosomes binding on one bead follows the Poisson statistics. Therefore, when the mean number of exosomes captured by each bead is smaller than 0.1, most beads (>99.53%) capture at most one target exosome. Therefore, 10× more beads were added than the expected exosomes to ensure single-exosome capture. To prove the successful capture of exosomes via CD63 antibody-antigen binding on beads, TEM experiments for were carried out. The magnetic beads coated with CD63 capture antibody were exposed to two samples: one with MDA-MB-231 exosomes and the other without exosomes as the control group. FIG. 11a shows a bare bead without exosomes on the surface while FIG. 11b clearly shows that one exosome was constructed on a magnetic bead. These results demonstrated that the functionalized magnetic beads were able to bind the exosomes specifically in a single complex through ExoELISA. After single exosomes were captured on beads, anti-GPC-1, previously biotinylated with a biotin tag, were used as the detection antibody to bind GPC-1 protein marker on the membranes of the target exosomes. After forming immunocomplex on the beads, the detection antibody was further conjugated with an enzymatic reporter, β-Galactosidase, which catalyzes the fluorescein-di-β-D-galactopyranoside (FDG) substrate to produce a fluorescent signal for detection in the droplet microfluidic system.

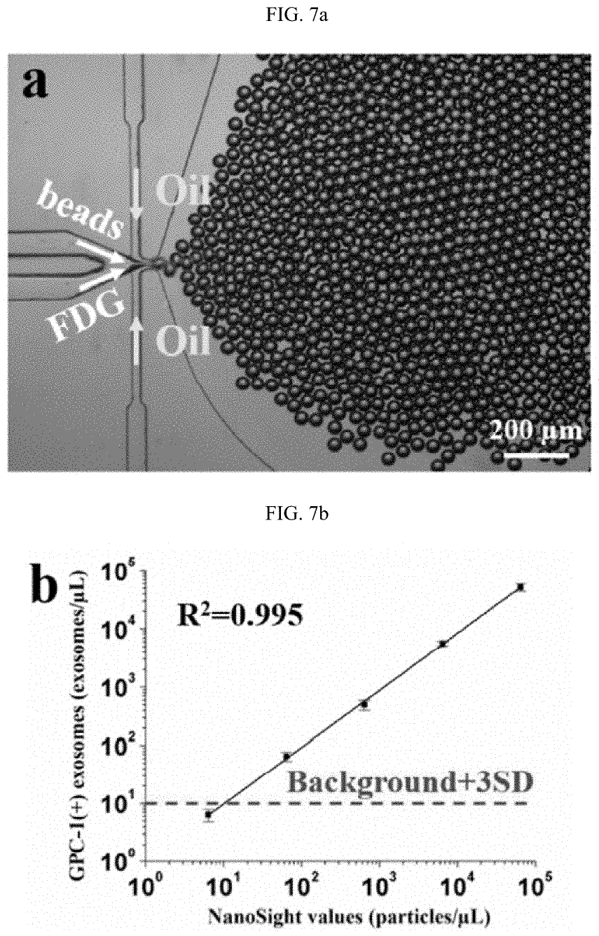

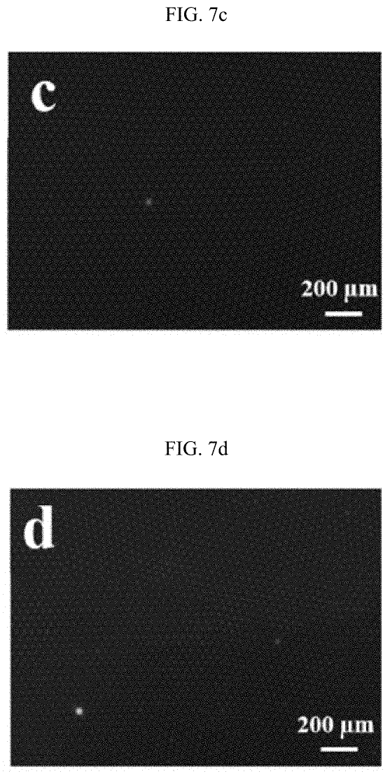

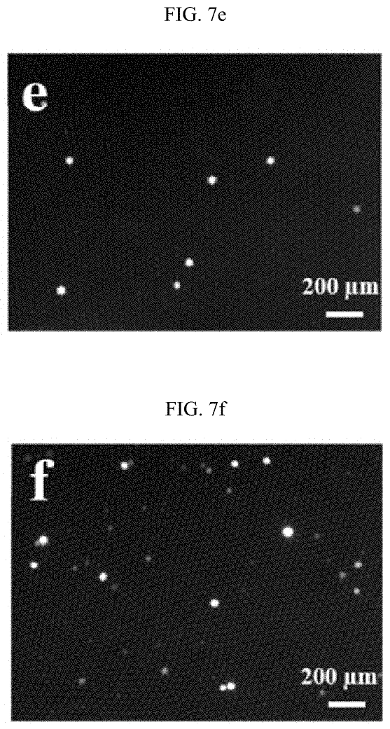

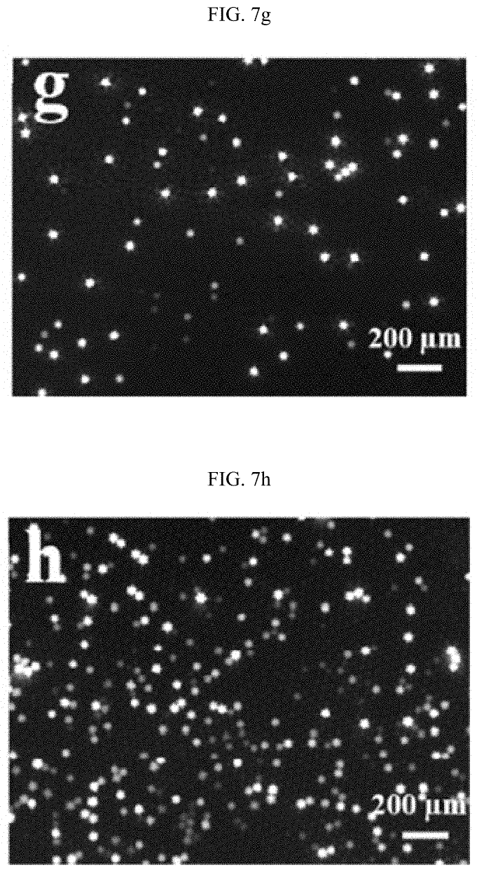

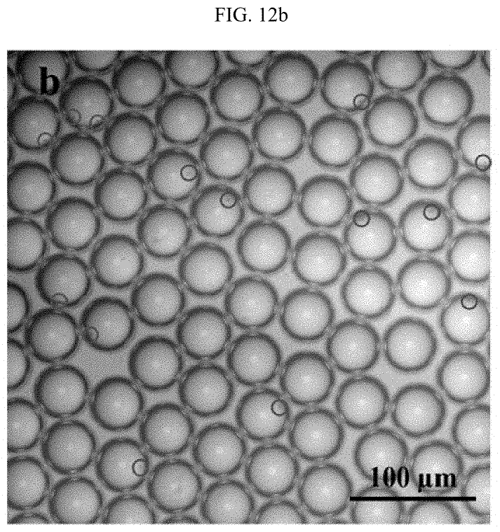

A flow-focusing droplet generation device with two sample inlets for the prepared bead sample and FDG substrate solutions respectively was used to generate droplets of 40 μm diameter in mineral oil (FIG. 7a). Likewise, the encapsulation of beads in microdroplets is also based on the Poisson distribution. The mean number of beads per droplet was set to be <0.3 to ensure most droplets contain none or one bead (see captured bright images of bead-encapsulated droplet arrays as examples in FIGS. 12a-12b). Importantly, the positive droplets that contain at least one target exosome can be calculated accordingly to the target molecule to magnetic bead ratio and the magnetic bead to droplet ratio following the 1 analysis of two dependent Poisson distribution. Both ratios were set sufficiently low to allow a linear dynamic range of Poisson statistics for counting the target exosomes. Therefore, almost all positive droplets only contained one target exosome. Also, direct “digital” counting of target exosomes was made feasible by simply counting the fluorescent droplets without the need of highly sensitive detection methods or complicated image processing for measuring the real number of the magnetic beads.

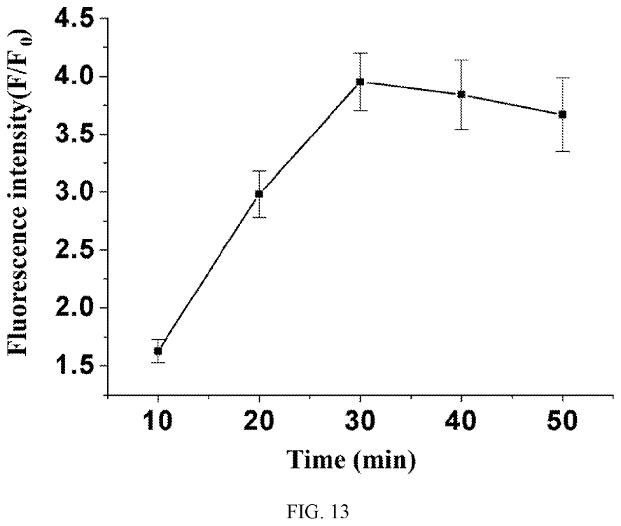

The produced droplets were spread in the droplet storage chamber in a single layer configuration and incubated before observation. The fluorescence signal rising time took a few minutes which suggested the effect of premixing in microchannels prior to droplet generation was negligible. The FDG catalysis reaction was investigated to optimize the assay incubation time (FIG. 13). 30 mins was chosen as the optimal incubation time for 40 μm diameter droplets, but a shorter time may be feasible if using smaller droplets. The end point counting of the fluorescent droplets (positive copies) was conducted once the incubation was completed. The number of fluorescent droplets represented the number of target exosomes.

Droplet digital ExoELISA was calibrated using the MDA-MB-231 exosomes mentioned above. A 10-fold serial dilution of the sample was conducted with an initial concentration of 6.39×10⁸ exosomes per mL. The results are shown in FIG. 7b. The detected GPC-1 exosomes were in an excellent linearity with the total particles measured in NanoSight. The error bars represent the standard deviation of three repeated experiments. Due to the picolitre droplet size, the LOD of our droplet digital ExoELISA, determined by the background (negative control) signal plus 3 times of standard deviation (SD) of the background signal, was approximately 10 exosomes/μL. Compared with the reported methods for detection of exosomes 2 (Table 1), the methods disclosed herein achieved the lowest LOD. Since in sample discretization a sufficient quantity of beads was mixed with exosomes and compartmentalized the beads into a sufficient number of droplets to achieve one fluorescent droplet representing one target exosome with over 99% confidence. FIG. 7c shows the background of the assay, possibly caused by non-specific binding to the surface of the beads or carry-through of free reporter enzymes into the encapsulated droplets. FIGS. 7d-7h are the images of the fluorescent droplets in the chamber by taking the 10-fold serial dilution. It is noted that among the fluorescent droplets, some droplets emitted stronger fluorescence signals than others. The variations could be due to various expressions of GPC-1 on a single exosome or the heterogeneous nature of single-enzyme catalysis. One million droplets were generated and the dynamic range was allowed to reach the range of 5 log of the linear regime. The dynamic range can be further extended by employing the two dependent Poisson statistics.

TABLE 1
Comparison of the limit-of-detection (LOD) and loading serum
sample volume of current assays for detection of exosomes.

| Detection | | LOD | Volume |
|---|---|---|---|
| platform | Assay | (particles/μL) | (μL) |
| Electro- | Electrochemical detection | 4.7 × 10 | 5 |
| chemistry | Electrochemical sandwich | 2 × 10 | 1.5 |
| | immunosensor | | |
| | Aptamer-based | 1 × 10 | Not |
| | electrochemical biosensor | | given |
| | Electrochemical sensor | 50 | 25 |
| | Integrated magneto- | 3 × 10 | 100 |
| | electrochemical exosome | | |
| | (iMEX) sensor | | |
| | Quantum dotbased | 100 | 10 |
| | sensitive detection | | |
| | Single Particle | 3.94 × 10 | 20 |
| | Interferometric Reflectance | | |
| | Imaging Sensor (SP-IRIS) | | |
| Optical | Surface-enhanced Raman | 1.2 × 10 | 100 |
| | scattering (SERS) | | |
| | nanoprobes | | |
| | Colorimetric aptasensor | 5.2 × 10 | Not |
| | | | given |
| | Integrated micorfluidics | 1 × 10 | 30 |
| Microfluidics | Immuno-capture on | 50 | 20 |
| | GO/PDA nano-interface | | |
| ExoELISA | Latera flow immunoassay | 8.54 × 10 | 100 |
| | (LFIA) | | |
| Microfluidics | Droplet digital ELISA (this | 10 | 10 |
| | work) | | |

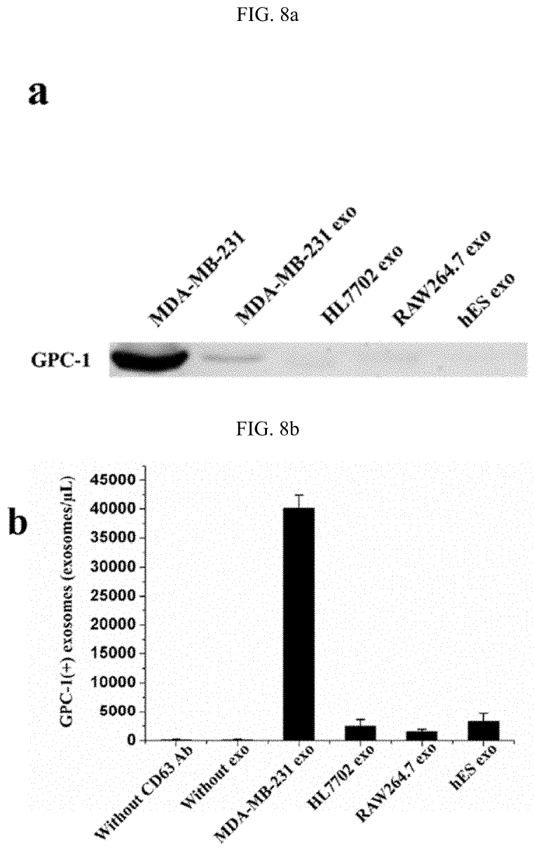

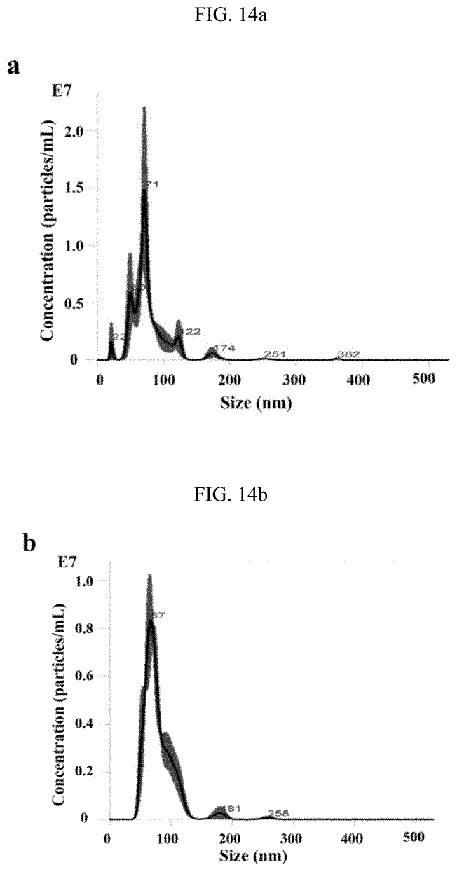

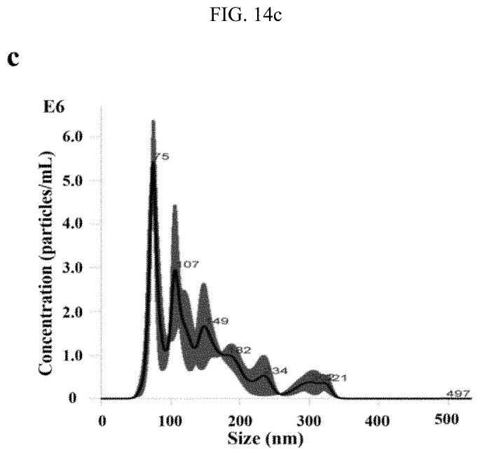

The variety of exosome subpopulation protein biomarkers significantly complicates exosome counting. The differentiation of exosome subpopulations is based on immunoassay, which possesses excellent specificity. To check the specificity of GPC-1(+) exosome detection in breast cancer exosomes (MDA-MB-231 exo), control experiments were performed using three kinds of non-cancerous exosomes including human normal liver exosomes (HL-7702 exo), mouse normal macrophage exosomes (RAW264.7 exo), and human embryonic stem exosomes (hES exo). Western blot analysis was used to identify the expression levels of GPC-1 in MDA-MB-231 exo, HL-7702 exo, RAW264.7 exo, and hES exo, and found that the expression of GPC-1 in MDA-MB-231 exo was slightly higher than the other three groups (FIG. 8a). Due to the limited detection capacity of western blot, if the sample contains a small amount of GPC-1(+) exosomes, other proteins on the exosomes in the sample may interfere with the GPC-1(+) in western blot analysis. Moreover, the western blot analysis can only qualitatively indicate whether GPC-1 is expressed in the sample as it cannot measure the specific number of GPC-1(+) exosomes. Next, the specificity of the droplet digital ExoELISA for GPC-1(+) exosome detection was measured among the four chosen exosomes and two negative controls: a sample using magnetic beads without CD63 Ab and a sample with no exosomes (FIG. 8b). NTA analysis was used to estimate the exosome number concentrations. The measured values were 4.22×10⁸, 2.86×10⁸, and 2.85×10⁸ particles per mL for HL-7702 exo, RAW264.7 exo, and hES exo, respectively (FIGS. 14a-14c). After proper dilution, each sample contained 6.39×104 15 exosomes per μL. Among these samples, only MDA-MB-231 exo showed significantly high number of GPC-1(+) exosomes (40141 exosomes per μL). For the negative control cases, very few fluorescent droplets were observed per experiment (˜5 detectable copies per μL), confirming the background of the assay is mainly due to the low enzyme non-specific binding to the magnetic beads.

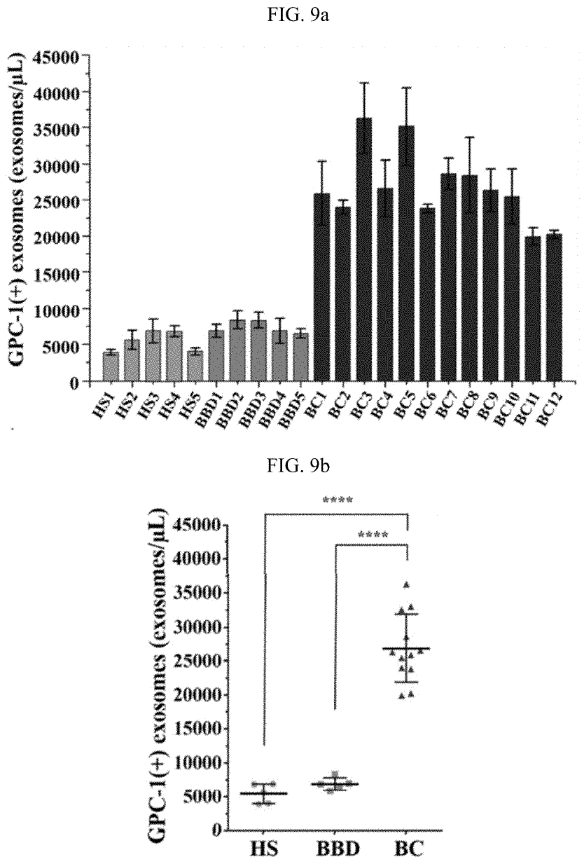

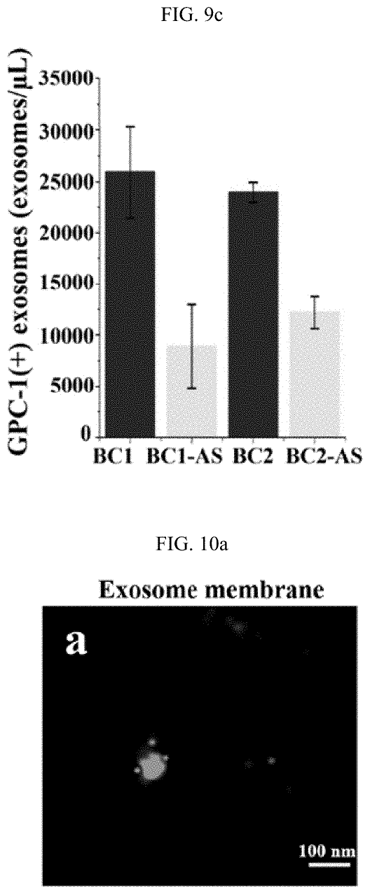

To demonstrate a clinically relevant application of our approach, the droplet digital ExoELISA was performed for detection of GPC-1(+) exosomes using clinical samples from serum of 5 healthy individuals (HS), 5 patients with benign breast disease (BBD), 12 patients with breast cancer 12 (BC), and 2 patients with breast cancer after surgery (BC-AS) (FIGS. 9a-9c). Serum samples obtained from HS were used as the control for this study. There are about 0.3%-4.7% (average of 2.3%) GPC-1(+) exosomes even in healthy human serum samples, and around 10′ vesicles per mL in blood. FIG. 9a shows that there was an average of 5448 GPC-1(+) exosomes per microliter in HS and similar GPC-1(+) exosomes (˜6914 exosomes/μL) in BBD, while the average GPC-1(+) exosomes in the BC group increased by five to seven fold. Thus, the expression of GPC-1 significantly increased on tumor-derived exosomes as compared to the normal and benign breast disease samples. The increase may be a result of a higher level of GPC-1(+) exosomes shed by tumor cells than normal cells. FIG. 9b shows that the BC patients overexpressed GPC-1(+) exosomes and can be well discriminated from the HS and BBD groups (p<0.0001). Notably, for BC1-AS and BC2-AS, two samples of patients BC1 and BC2 after surgery, the measured values of GPC-1(+) exosomes in BC1-AS and BC2-AS were significantly lower than BC1 and BC2 (FIG. 5c), respectively, but relatively higher than HS and BBD (FIG. 5a). Therefore, these data not only verified the GPC-1 can be regarded as an exosomal biomarker to distinguish non-BC subjects from patients with breast cancer, but also suggested that the methods disclosed herein are suitable for detection of GPC-1(+) exosomes for pre- and post-surgical monitoring. The droplet digital ExoELISA has been demonstrated as a reliable method for quantifying target exosomes from HS, BBD, and BC-AC from BC clinical samples. In the early stage of the diseases (especially cancer), where some exosome subpopulations only secreted by tumor cells are extremely small, the droplet digital ExoELISA can be extremely valuable for detecting the extremely low abundance exosomes than other reported methods (Table 1). Therefore, the droplet digital ExoELISA can be used for early cancer diagnostics and post-surgical monitoring in clinical research.

The disclosure describes methods to leverage the droplet microfluidics for single molecule/copy detection. The standard ExoELISA techniques were extended for detection of ultralow ambulance exosomes with specific target proteins. The digital ExoELISA method is able to achieve unprecedented accuracy and high specificity for exosome quantification, and can distinguish the target protein expression level on single exosomes through the fluorescence signal level in droplets. The droplet digital ExoELISA can detect the target exosomes in a dynamic range of 5 log and the detection limit can be as few as 10 exosomes per μL. The high specificity was also demonstrated by quantifying the exosomes with target GPC-1 biomarker from a variety of exosome subpopulation protein biomarkers. The methods disclosed herein can be used for absolute quantification of exosomes in serum samples from breast cancer patients. Thus, the droplet digital ExoELISA method can propel the discovery of cancer exosomal biomarkers.

Methods

Microfluidic Device Fabrication and ExoELISA Assays in Microdroplets

The droplet digital ExoELISA devices were made of polydimethylsiloxane (PDMS) using standard soft lithography procedures. Sylgard-184 PDMS (Dow Corning) in 10:1 mixing ratio of base and cross-linker was cast on top of the master mold, degassed in a vacuum and cured in an oven at 70° C. for two hours. Afterwards, the cured PDMS was released from the mold and cut into individual chips. The access holes for liquid inlet and outlet were punched using a pan needle. The PDMS replica and a glass slide (SAIL BRAND) were treated with Oz plasma and bonded together. The devices were baked on a hot plate at 100° C. for 8 hours to recover the surface hydrophobicity. The magnetic bead and fluorescein-di-β-D-galactopyranoside (FDG) substrate solution was encapsulated into 40 μm diameter droplets by mineral oil with 3 wt. % ABIL EM 90 and 0.1 wt. % Triton X-100 stabilizing surfactants (FIG. 7a). For device operation, the flow rates of the bead suspension and FDG phase were kept identical at 0.7 μL/min while the flow rate of oil phase was controlled at 2.3 μL/min using a syringe pump (PHD ULTRA, Harvard Apparatus). After the droplet generation was accomplished, the droplets were incubated in situ for 30 minutes.

Fluorescence Image Acquisition and Data Analysis

After the completion of incubation, the device was placed on an inverted epifluorescent microscope (Eclipse Ti-U, Nikon) with a fiber illuminator (Nikon Intensilight C-HGFI) at an intensity of 50 mW through a filter cube for FITC 18 dye (Ex. 490 nm, Em: 525 nm). To alleviate the complexity and duration of the droplet imaging process, the whole droplet storage chamber was scanned on an automatic XY motorized stage, the images were taken using a CCD camera (EXi Blue, QImaging) coupled with a 2× objective to have a wider image window for counting more droplets in one frame. After all the images of droplets in the storage chamber were taken, a custom-made program was used to merge and analyze the fluorescent and total droplets. By setting the intensity threshold, two distinct droplet populations were obtained with different intensity and count the positive droplet numbers. In each experiment, one million droplets were counted for data analysis.

Cell Culture and Exosome Isolation

All the cell lines were obtained from Cell Bank of the Chinese Academy of Sciences, Shanghai, China. MDA-MB-231 and HL-7702 were cultured in 5 RPMI-1640 medium containing 10% (v/v) fetal bovine serum (FBS, System Biosciences) and 61% (v/v) penicillin-streptomycin. RAW264.7 was cultured in DMEM cell culture medium, supplemented with 10% (v/v) FBS, and 1% (v/v) penicillin-streptomycin. All cell lines were incubated in a humidified atmosphere of 5% CO₂ at 37° C. For the isolation of exosome from the three cell lines, the cells were cultured in media with 10% (v/v) FBS and 1% (v/v) penicillin-streptomycin to 60-70% confluency, washed twice with phosphate buffer solution (PBS), then maintained for 12 h in serum-free basal media, then washed once with PBS, and then maintained for 48 h in media with 2% (v/v) Exo-FBS™ exosome-depleted FBS (System Biosciences) and 1% (v/v) penicillin-streptomycin. hES (Human embryonic stem) cell line was cultured in PSCeasy medium (Cellapybio) at 37° C. in a 5% CO₂ incubator to 90-100% confluency. Supernatants were collected from the four cell lines and sequentially centrifuged at 2000 g for 20 min to eliminate cells and debris and at 10000 g for 30 min to eliminate microvesicles. Then, exosomes were ultra-centrifugated twice using a W32Ti rotor (L-80XP, Beckman Coulter) at 135000 g for 70 min and resuspended in PBS and stored at −80° C. till further use.

Nanoparticle Tracking Analysis (NTA)

The concentration and size of exosomes were measured using a NanoSight NS300 and NTA 3.2 software (Malvern). Samples were diluted to suitable concentrations ˜1×10⁷-10⁹ particles/mL and injected in a detection chamber equipped with a 405 nm laser. Three sets of measurements were performed, each lasting 60 sec.

Dual-Color Super-Resolution Imaging

50 μL of exosome sample solution was fixed on a coverslip (SALD BRAND) coated by Poly-L-lysine (Sigma-Aldrich), incubated for 30 min at room temperature, and then washed three times with PBS. The exosome membranes were stained using a PKH67 Green Fluorescent Cell Linker Mini Kit (Sigma-Aldrich). 50 μL of PKH67 diluted solution was rapidly applied to the sample, and mixed by pipetting. The mixture was incubated for 4 min with periodic mixing at room temperature, then 100 μL of 1% BSA was added for 2 min to inhibit binding of excess dyes. After rinsing with PBS three times, the coverslip was immediately placed into the primary antibody solution (either 1:400 anti-CD63 or 1:400 anti-GPC-1) for 1 h at room temperature, then washed three times with PBS. In the last step, Alexa Fluor 647-conjugated secondary antibody (1:2000 Bioss, bs-0295G-AF647) was applied, followed by 30 min incubation at room temperature. The final sample was washed three times with PBS and stored in PBS for further super-resolution imaging of exosomes.

A Nikon N-STROM (stochastic optical reconstruction microscopy) super-resolution microscope system was used to capture images through total internal reflection fluorescence 14 (TIRF) illumination with 488- and 647-nm. During imaging, the exosomes were immersed in an imaging buffer which was composed of 0.56 mg/mL glucose oxidase (Sigma-Aldrich), 0.3 mg/mL catalase (Sigma-Aldrich), and 10 mM cysteamine (Sigma-Aldrich) in PBS. PKH67 and Alexa Fluor 647 conjugated on the second antibody were excited for imaging of the exosome membranes and proteins (either CD63 or GPC-1), respectively. A series of 20000 images were acquired by an iXon3 DU-897E electron-multiplying charge-coupled device (EMCCD) camera (Andor Technology) through a Plan Apochromat TIRF 100× oil immersion lens with numerical aperture of 1.49.

Transmission Electron Microscope (TEM)

The isolated exosomes were stained with 2% phosphotungstic acid (PTA) with a concentration ratio of 4:1 for 10 min. The mixtures were then loaded onto copper grids and left to dry at room temperature. The grids observed with transmission electron microscope (HITACHI H-7650). For TEM analysis of immunomagnetic captured exosomes, the single-exosome-bead complexes were prepared using CD63-coated magnetic beads according to the Poisson distribution. The mixture was then stained with 2% PTA for 10 min and placed on a copper grid. After further drying, the grid was imaged by TEM. The CD63-coated magnetic beads without mixing with exosomes were used as a negative control.

Western Blot Analysis

Total protein from MDA-MB-231 cells were extracted by RIPA lysis buffer (Beyotime Institute of Biotechnology). The cell proteins or exosome supernatants were denatured in 5× sodium dodecyl sulfonate (SDS) buffer. 20 μg protein per lane were separated by 10% SDS-polyacrylamide gel electrophoresis and transferred onto the polyvinylidene difluoride (PVDF) membranes (Millipore, Billerica), blocked in 5% skimmed milk for 2 h at room temperature, followed by washing three times with TBS-Tween 20 (TBST) buffer (137 mM NaCl, 25 mM Tris-HCl, pH 7.6, 0.1% Tween 20). The membranes were probed with 1:1000 anti-CD63 (ab134045, Abcam) or 1:1000 anti-GPC-1 (ab199343, Abcam) overnight at 4° C. After washing with TBST buffer, blots were incubated with a fluorescent secondary antibody (Cell Signaling Technology) for 1 h at room temperature, followed by chemiluminescence measurement with Bio-Rad ChemiDoc XRS Imager system (Bio-Rad Laboratories).

Preparation of Magnetic Beads Conjugated with CD63 Antibody

The antibody-conjugated magnetic beads were prepared with Dynabeads® MyOne™ carboxylic acid (Invitrogen, Life Technology) according to the manufacturer's instructions. Briefly, the carboxylic acid group on the magnetic beads was activated by N-Ethyl-N′-(3-dimethylaminopropyl) carbodiimide hydrochloride (EDC, Thermo Scientific), then a volume of 50 μL activated magnetic beads were mixed with 10 μl of CD63 antibody. Beads were blocked with 0.1% Bovine serum albumin (BSA, Sigma-Aldrich), washed several times with PBS, then resuspended in 100 μL of PBS before use. The final concentration of CD63-coated magnetic beads was estimated as 3.5-6.0×10⁶ beads/μL according to the initial concentration.

Modification of GPC-1 Antibody with Biotin Tag

The biotinylation of anti-GPC-1 was performed using a EZ-Link® Micro Sulfo-NHS-LC-Biotinylation Kit (Thermo Scientific). 10 μL of anti-GPC-1 with 0.24 μL of 9 mM Sulfo-NHS-LC-Biotin was combined at room temperature for 1 h. Then the excess biotin was removed using Zeba desalting columns (Thermo Scientific), which yielded 400 μL of 1:40 biotinylated anti-GPC-1 for the next study.

Exosome Capture, Magnetic Isolation, and Enzyme Conjugation

CD63-functionalised magnetic beads were mixed with MDA-MB-231 exosomes (at various concentrations of 6.39, 63.9, 639, 6390, 63900 particles/μL). The mixture was incubated for 1 h in HulaMixer® Sample Mixer (Invitrogen, Life Technology) with periodic mixing at room temperature to allow the antibody to capture the exosome targets. The beads were isolated by a magnet for 2 min and washed with PBS three times. Next, 40 μL of 1:400 biotinylated anti-GPC-16 was added and the resultant mixture was incubated in a mixer for 1 h at room temperature, followed by isolation by a magnet for 2 min and washing by PBS three times. In the final step, 40 μL of 2 ng/μL β-Galactosidase (Invitrogen, Life Technology) was mixed with immunomagnetic captured exosomes and incubated for 30 min at room temperature, then washed with PBS three times and resuspended in 15 μL of PBS for further application on chip.

Clinical Sample Preparation

A total of 24 clinical serum samples (5 HS, 5 patients with 22 BBD, 12 patients with BC and 2 patients with BC-AS) were obtained from the Department of Laboratory Medicine, Nanfang Hospital, Southern Medical University, Guangzhou, China. The diagnoses of BBD and BC were confirmed by histological examination of tissue biopsy. The serum samples were centrifuged twice at 2000 g for 5 min to eliminate cells and debris, then at 16100 g for 20 min to remove microvesicles. The supernatants were carefully collected and stored at −80° C. prior to use. The involved clinical serum samples were approved by the ethics committee of Nanfang Hospital, Southern Medical University, and written consents were obtained from all patients and healthy individuals.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Liu C., Xu X., Li B., Situ B., Pan W., Hu Y., An T., Yao S., and Zheng L., “Single-exosome-counting immunoassays for cancer diagnostics,” Nano Letters, 2018, 18, 7, 4226-4232.
2. Raposo, G. A.; Stoorvogel, W., Extracellular vesicles: Exosomes, microvesicles, and friends. J Cell Biol. 2013, 200, 373-383.
3. Melo, S. A.; Luecke, L. B.; Kahlert, C.; Fernandez, A. F.; Gammon, S. T.; Kaye, J.; LeBleu, V. S.; Mittendorf, E. A.; Weitz, J.; Rahbari, N.; Reissfelder, C.; Pilarsky, C.; Fraga, M. F.; Piwnica-Worms, D.; Kalluri, R., Glypican-1 identifies cancer exosomes and detects early pancreatic cancer. Nature 2015, 523, 177-182.
4. Zhao, Z.; Yang, Y.; Zeng, Y.; He, M., A microfluidic ExoSearch chip for multiplexed exosome detection towards blood-based ovarian cancer diagnosis. Lab Chip 2016, 16, 489-496.
5. An, T.; Qin, S., Xu, Y., Tang, Y.; Huang, Y.; Situ, B.; Inal, J. M.; Zheng, L., Exosomes serve as tumour markers for personalized diagnostics owing to their important role in cancer metastasis. J. Extracell. Vesicles 2015, 4, 27522.
6. Contreras-Naranjo, J. C.; Wu, H.; Ugaz, V. M., Microfluidics for exosome isolation and analysis: enabling liquid biopsy for personalized medicine. Lab Chip 2017, 17, 3558-3577.
7. Jeong, S.; Park, J.; Pathania, D., Castro, C. M.; Weissleder, R., Lee, H., Integrated Magneto-Electrochemical Sensor for Exosome Analysis. ACS Nano 2016, 10, 1802-1809.
8. Shim, J. U.; Ranasinghe, R. T.; Smith, C. A.; Ibrahim, S. M.; Hollfelder, F.; Huck, W. T.; Klenerman, D.; Abell, C., Ultrarapid generation of femtoliter microfluidic droplets for single-molecule-counting immunoassays. ACS Nano 2013, 7, 5955-5964.
9. Rissin, D. M.; Kan, C. W.; Campbell, T. G.; Howes, S. C.; Fournier, D. R.; Song, L.; Piech, T.; Patel, P. P.; Chang, L.; Rivnak, A. J.; Ferrell, E. P.; Randall, J. D.; Provuncher, G. K.; Walt, D. R.; Duffy, D. C., Single-molecule enzyme-linked immunosorbent assay detects serum proteins at subfemtomolar concentrations. Nat. Biotechnol. 2010, 28, 595-599.
10. Scheler, O.; Pacocha, N.; Debski, P. R.; Ruszczak, A.; Kaminski, T. S.; Garstecki, P., Optimized droplet digital CFU assay (ddCFU) provides precise quantification of bacteria over a dynamic range of 6 logs and beyond. Lab Chip 2017, 17, 1980-1987.
11. Amselem, G.; Guermonprez, C.; Drogue, B.; Michelin, S.; Baroud, C. N., Universal microfluidic platform for bioassays in anchored droplets. Lab Chip 2016, 16, 4200-4211.
12. Chang, L.; Rissin, D. M.; Fournier, D. R.; Piech, T.; Patel, P. P.; Wilson, D. H.; Duffy, D. C., Single molecule enzyme-linked immunosorbent assays. Theoretical considerations. J. Immunol. Methods 2012, 378, 102-115.
13. Guan, W.; Chen, L.; Rane, T. D.; Wang, T., Droplet Digital Enzyme-Linked Oligonucleotide Hybridization Assay for Absolute RNA Quantification. Sci. Rep. 2015, 5.
14. Lui, C.; Xu, X.; Li, B.; Situ, B.; Pan, W.; Hu, Y.; An, T.; Yao, S.; and Zheng, L.; Single-exosome counting immunoassays for cancer diagnostics; Nano Letter; 2018.

1. A method for quantifying exosomes in a sample, comprising:
a) contacting a sample containing a plurality of exosomes with:
i) a capture bead comprising a bead conjugated to a first binding agent, and
ii) a second binding agent comprising a detectable label,
wherein the first binding agent specifically binds to a first biomolecule present in the plurality of exosomes to produce a first complex comprising the capture bead and a first exosome, the second binding agent specifically binds to a second biomolecule present in the plurality of exosomes to produce either an exosome-second binding agent complex comprising the second binding agent and a second exosome or a second complex comprising the capture bead, the first exosome, and the second binding agent;
b) from the composition produced at the end of step a), separating the capture beads, the first complexes, and the second complexes;
c) from the composition produced at the end of step b), separating from each other each of the capture beads, the first complexes, and the second complexes;
d) optionally, contacting the separated capture beads, the first complexes, and the second complexes with a substrate that produces a detectable signal from the second binding agent present in the second complexes; and
e) detecting the detectable signal from the second complexes to quantify the exosomes in the sample.

2. The method of claim 1, wherein the step a) comprises:
i) contacting the sample simultaneously with the capture bead and the second binding agent,
ii) contacting the sample with the second binding agent first, followed by contacting with the capture bead, or
iii) contacting the sample with the capture bead first, followed by contacting with the second binding agent.

3. The method of claim 1, wherein the bead has a diameter of 4 to 5 microns.

4. The method of claim 1, wherein the bead comprises agarose, an inert polymer, a superparamagnetic material, or any combination thereof.

5. The method of claim 1, wherein the bead comprises ferrite or magnetite (Fe₃O₄), optionally coated with polystyrene.

6. The method of claim 1, wherein each of the first binding agent and the second binding agent is, independent of each other, an antibody, an antigen binding fragment of an antibody, an aptamer, a protein binding partner, or a nucleic acid binding partner.

7. The method of claim 1, wherein each of the first binding agent and the second binding agent, independently of each other, specifically binds to CD9, CD63, CD81, GPC1, FN, PSMA, or microRNA-145.

8. The method of claim 1, wherein the step of separating from each other the capture beads, the first complexes, and the second complexes comprises a droplet generation.

9. The method of claim 8, wherein the droplet generation comprises an active droplet generation.

10. The method of claim 8, wherein the droplet generation comprises a passive droplet generation.

11. The method of claim 10, wherein the passive droplet generation comprises a cross-flowing droplet generation, flow focusing droplet generation, or co-flowing droplet generation.

12. The method of any of claim 1, wherein the step of separating the capture beads, the first complexes, and the second complexes comprises separating the beads into microwells.

13. The method of claim 12, wherein separating the beads into the microwells comprises introducing the composition produced at the end of step b) onto a support comprising the microwells.

14. The method of claim 13, wherein the microwells have a size of about 500 nl and the support comprises poly(dimethylsiloxane) polymer or a glass bottom bonded to a silicon grid that creates the microwells.

15. The method of claim 1, wherein the detectable labels is a fluorescent moiety, chemiluminescent reagent, bioluminescent reagent, enzyme, or radioisotope.

16. The method of claim 1, wherein the detectable label is an enzyme and the method comprises contacting the composition produced at the end of step b) with a substrate for producing the detectable signal.

17. The method of claim 1, wherein detecting the signal from the second complexes comprises: imaging with a camera the support comprising the microwells containing the capture beads, the first complexes, and the second complexes; or fluorescent sorting of the droplets comprising the capture beads, the first complexes, and the second complexes.

18. A method of detecting a cancer in a subject, the method comprising:
(I) determining the level of exosomes containing one or more cancer biomarkers in:
i) a test sample obtained from the subject, and
ii) optionally, a control sample;
(II) optionally obtaining a reference value corresponding to the level of exosomes containing one or more cancer biomarkers, (III) identifying the subject as:
i) having the cancer based on the level of exosomes containing one or more cancer biomarkers in the test sample compared to the level in the control sample or the reference value, or
ii) not having the cancer based on the level of exosomes containing one or more cancer biomarkers in the test sample compared to the level in the control sample or the reference value.

19. The method of claim 18, wherein the method for determining the level of exosomes containing one or more cancer biomarkers in a sample, comprises the steps of:
a) contacting the sample with:
i) a capture bead comprising a bead conjugated to a first binding agent, and
ii) a second binding agent comprising a detectable label,
wherein the first binding agent specifically binds to a first cancer biomarker present in the exosomes to produce a first complex comprising the capture bead and a first exosome, the second binding agent specifically binds to a second cancer biomarker present in the exosomes to produce either an exosome-second binding agent complex comprising the second binding agent and a second exosome or a second complex comprising the capture bead, the first exosome, and the second binding agent;
b) from the composition produced at the end of step a), separating the capture beads, the first complexes, and the second complexes,
c) from the composition produced at the end of step b), separating from each other each of the capture beads, the first complexes, and the second complexes,
d) optionally, contacting the separated capture beads, the first complexes, and the second complexes with a substrate that produces a detectable signal from the second binding agent present in the second complexes,
e) detecting the detectable signal from the second complexes to quantify the exosomes in the sample.

20. The method of claim 18, wherein each of the first binding agent and the second binding agent is, independently of each other, an antibody, an antigen binding fragment of an antibody, an aptamer, a protein binding partner, or a nucleic acid binding partner of CD9, CD63, CD81, GPC1, FN, PSMA, or microRNA-145.
